(19) United States
(12) Patent Application Publication
(10) Pub. No.: US 2012/0184457 A1
(43) Pub. Date: Jul. 19, 2012

(54) Modulation of miRNA Activity

(75) Inventors: Eric Miska, Cambridge (GB); Nicolas Lehrbach, Cambridge (GB); Javier Armisen-Garrido, Cambridge (GB); Shankar Balasubramanian, Cambridge (GB)

(73) Assignee: Cambridge Enterprise Limited, Cambridge (GB)

(21) Appl. No.: 13/389,159

(22) PCT Filed: Aug. 6, 2010
(86) PCT No.: PCT/GB2010/001492

§ 371 Date: Feb. 6, 2012

(30) Foreign Application Priority Data
Aug. 6, 2009 (GB) 0913752.2
Aug. 6, 2009 (GB) 0913753.0

(51) Int. Cl. G01N 33/53; C12Q 1/68; C40B 30/04

(52) U.S. Cl. 506/9; 436/501; 435/6.12

(57) ABSTRACT

Provided is a method of identifying a compound which modulates miRNA activity comprising (i) determining the ability of a test compound to alter the polyuridylation activity of a ZCCHC polypeptide wherein a test compound which alters the polyuridylation activity is useful in modulating miRNA activity; or (ii) determining the ability of a test compound to alter the binding of a ZCCHC polypeptide to a LIN28 polypeptide, wherein a test compound which alters said binding may be useful in modulating miRNA activity; or (iii) determining the ability of a test compound to bind to a ZCCHC polypeptide, wherein a test compound which binds to the ZCCHC polypeptide may be useful in modulating miRNA activity.

### PRIORITY

This application claims priority to GB 0913752.2 filed on 6 Aug. 2009 and GB 0913753.0 filed on 6 Aug. 2009, and the contents of both are incorporated by reference herein in their entirety.

### FIELD OF THE INVENTION

This invention relates to the modulation of miRNA activity in cells.

### BACKGROUND

Short RNAs have recently emerged as abundant regulators of gene expression in many eukaryotes including plants, animals and fungi. miRNAs are a class of ˜22 nucleotide (nt) RNAs that modulate gene expression by blocking translation and/or destabilizing target mRNAs^{11,12}. In animals, miRNAs are transcribed as long RNA precursors (pri-miRNAs), which are either processed in the nucleus by the RNase III enzyme complex Drosha-Pasha/DGCR8 to form ˜80 nt pre-miRNAs or are derived directly from intons¹³. pre-miRNAs are exported from the nucleus and processed by the RNase III enzyme Dicer, and incorporated into an Argonaute-containing RNA induced silencing complex (RISC).

The first identified miRNAs, the products of the C. elegans genes lin-4 and let-7, control cell fates during larval development¹⁴. When lin-4 or let-7 is inactivated, specific epithelial cells undergo additional cell divisions instead of their normal differentiation. lin-4 acts during early larval development by negatively regulating the lin-14 and lin-28 mRNAs¹⁴⁻¹⁸. The let-7 miRNA acts during late larval development and regulates lin-41, hbl-1, daf-12 and pha-4 mRNAs¹⁹⁻²². As such, the time of appearance of these miRNAs must be tightly controlled during development.

Numerous miRNAs have now been identified in a range of organisms, including humans. miRNAs display a number of different cellular functions and have been associated with a number of disease conditions in humans, including cancer and pathologies, including neurodegenerative conditions such as Alzheimer's disease and Parkinson's disease, viral infections, diabetes, and myopathies⁵⁰⁻⁵³.

Posttranscriptional regulation of specific miRNAs has recently been uncovered²³. For example, let-7 biogenesis has been shown to be blocked by abnormal cell LINeage family member-28 (LIN28) at either the Drosha^{7.9} or Dicer^{6,10} step in mammalian cell culture.

The present invention relates to the finding that the microRNA processing is regulated by a poly-(U) polymerase (ZCCHC). Modulation of this poly-(U) polymerase may be useful in modulating miRNA activity. The ZCCHC poly-(U) polymerase may therefore represent a useful a target for therapeutics, for example, to modulate miRNA-mediated cellular activities, such as proliferation and differentiation.

### SUMMARY OF THE INVENTION

In various aspects, the invention provides methods of identifying compounds which modulate the miRNA activity by altering the poly (U) polymerase mediated processing of pre-miRNA.

### DETAILED DESCRIPTION OF THE INVENTION

An aspect of the invention provides a method of identifying a compound which modulates miRNA activity comprising;
determining the ability of a test compound to alter the polyuridylation activity of a ZCCHC polypeptide,
wherein a test compound which alters the polyuridylation activity may be useful in modulating miRNA activity.

The ability of the test compound to alter the polyuridylation activity of a ZCCHC polypeptide may be determined by contacting the ZCCHC polypeptide and an RNA molecule in the presence and absence of the test compound and determining or measuring the amount of polyuridylation of the RNA molecule by the ZCCHC polypeptide.

A difference in the polyuridylation of the RNA molecule in the presence relative to the absence of the test compound is indicative that the test compound alters the polyuridylation activity of ZCCHC and may therefore be useful in the modulation of miRNA activity.

Suitable RNA molecules include pre-miRNA molecules. In some preferred embodiments, the RNA molecule may be a let-7 pre-miRNA.

Suitable let-7 pre-miRNAs may include any eukaryotic pre-let-7 miRNA, such as C. elegans pre-let-7, or a mammalian pre-let-7, such as human pre-let-7 or variants or mimetics thereof. A let-7 pre-miRNA may include any member of the let-7 family. For example, the polyuridylation of a pre-miRNA selected from the group consisting of cel-let-7 (MI0000001), hsa-let-7a-1 (MI0000060), hsa-let-7a-2(MI0000061), hsa-let-7a-3(MI0000062), hsa-let-7b (MI0000063), has-let-7c (MI0000064), hsa-let-7d (MI0000065), hsa-let-7e (MI0000066), hsa-let-7f-1 (MI0000067), hsa-let-7f-2 (MI0000068), hsa-let-7g (MI0000433) and/or hsa-let-7i (MI0000434) may be determined.

A compound identified by a method described herein may modulate the activity of any miRNA. In some preferred embodiments, the activity of an miRNA of the let-7 family may be modulated. A suitable let-7 miRNA may include the mature form of a pre-miRNA described above, for example the activity of cel-let-7 (MIMAT0000001), hsa-let-7a (MIMAT0000062), hsa-let-7a* (MIMAT0004481), hsa-let-7a-2* (MIMAT0010195), hsa-let-7b (MIMAT0000063), hsa-let-7b* (MIMAT0004482), hsa-let-7c (MIMAT0000064), hsa-let-7c* (MIMAT0004483), hsa-let-7d (MIMAT0000065), hsa-let-7d* (MIMAT0004484), hsa-let-7e (MIMAT0000066), hsa-let-7e* (MIMAT0004485), hsa-let-7f (MIMAT0000067), hsa-let-7f-1* (MIMAT0004486), hsa-let-7f-2* (MIMAT0004487), hsa-let-7g (MIMAT0000414), hsa-let-7g* (MIMAT0004584), hsa-let-71 (MIMAT0000415), hsa-let-71*(MIMAT0004585) may be modulated.

The sequences of eukaryotic pre-miRNAs and miRNAs are publically available, for example from the miRNA Registry (miRBase) which is maintained by the Wellcome Trust Sanger Institute, Hinxton, UK. The miRBase database is described in Griffiths-Jones S, et al Nucleic Acids Res. 2008 36:D154-D158; Griffiths-Jones S, NAR, 2004, 32, D109-D111 and Griffiths-Jones S et al NAR, 2006, 34, D140-D144) and is available online at http://microrna.sanger.ac.uk/.

In some embodiments, the RNA substrate may be immobilised on a solid support, for example in a high-throughput scintillation proximity or filter binding assay.

Polyuridylation by a ZCCHC polypeptide may be determined in the presence of a LIN-28 polypeptide. The ability of the test compound to alter LIN-28 dependent polyuridylation may be determined by contacting a ZCCHC polypeptide, a LIN28 polypeptide and an RNA molecule in the presence and absence of the test compound and determining or measuring the polyuridylation of the RNA molecule.

A difference in the LIN-28 dependent polyuridylation of the RNA molecule in the presence relative to the absence of the test compound is indicative that the test compound alters the LIN28-dependent polyuridylation activity of the ZCCHC polypeptide and may therefore be useful in the modulation of miRNA levels or activity.

A range of suitable techniques which may be used to determine the polyuridylation activity of a ZCCHC polypeptide are known in the art. For example, the incorporation of labelled UTP into an RNA molecule may be determined using standard techniques. Suitable labels are known in the art and include radiolabels, such as ³²P (i.e. α-³²P-UTP), or fluorescent labels, such as fluorescein. Cy3, Cy5 or Alexa Fluor 546.

The amount of radiolabelled UTP which is incorporated into an RNA molecule by a ZCCHC polypeptide may be determined using conventional techniques, such as gel electrophoresis and phosphorimaging; or scintillation counting, or scintillation proximity assay.

The amount of fluorescent-labelled UTP which is incorporated into an RNA molecule by a ZCCHC polypeptide may be determined using conventional fluorescence-based techniques, such as FRET.

Other aspects of the invention relate to the identification of test compounds which bind to the ZCCHC poly(U)polymerase as candidate modulators of miRNA activity.

A method of identifying a compound which modulates miRNA activity may comprise:
determining the ability of a test compound to bind to a ZCCHC polypeptide,
wherein a test compound which binds to ZCCHC polypeptide may be useful in modulating miRNA activity.

A test compound which binds to ZCCHC polypeptide is a candidate modulator of miRNA activity, for example let-7 activity.

Suitable techniques for determining binding are described in more detail below.

Other aspects of the invention relate to the identification of test compounds which modulate the binding of the ZCCHC poly(U)polymerase to LIN28 as candidate modulators of miRNA activity.

A method of identifying a compound which modulates miRNA activity in cell may comprise;
determining the ability of a test compound to alter the binding of a ZCCHC polypeptide to a LIN28 polypeptide,
wherein a test compound which alters said binding may be useful in modulating miRNA levels or activity.

The ability of the test compound to alter binding may be determined by contacting the ZCCHC polypeptide and the LIN28 polypeptide in the presence and absence of the test compound and determining or measuring binding between the ZCCHC polypeptide and the LIN28 polypeptide.

A difference in the binding of the ZCCHC polypeptide to the LIN28 polypeptide in the presence relative to the absence of the test compound is indicative that the test compound may be useful in the modulation of miRNA levels or activity.

A test compound may reduce or inhibit the binding of the ZCCHC polypeptide to the LIN28 polypeptide. Pre-miRNA is targeted for degradation by LIN28-mediated polyuridylation by the ZCCHC poly(U)polymerase. Disruption of ZCCHC poly(U)polymerase binding to LIN28 reduces the polyuridylation of pre-miRNA and therefore increases the amount of pre-miRNA which is processed into active miRNA. A decrease in binding in the presence of the test compound relative to the absence may therefore be indicative that the test compound increases or promotes miRNA activity in a cell.

A method described herein, a test compound may be found to increase or enhance the binding of the ZCCHC polypeptide to the LIN28 polypeptide. Promotion of the binding of the ZCCHC poly(U)polymerase to LIN28 increases the polyuridylation of pre-miRNA and therefore reduces the amount of pre-miRNA which is processed into active miRNA. An increase in binding in the presence of the test compound relative to the absence may therefore be indicative that the test compound reduces miRNA activity in a cell.

The ZCCHC polypeptide and the LIN28 polypeptide may be contacted under conditions in which they bind together unless a compound which inhibits binding is present. Alternatively, the ZCCHC polypeptide and the LIN28 polypeptide may be contacted under conditions in which they do not bind together unless a compound which promotes binding is present.

A range of techniques known in the art may be used to determine the binding between a test compound and a ZCCHC polypeptide or between ZCCHC and LIN28 polypeptides, including scintillation proximity assays, flow cytometry (e.g. FACS), immunohistochemical or immunocytochemical staining, surface plasmon resonance (e.g. BIAcore™), Western Blotting, immunofluorescence, enzyme linked immunosorbent assays (ELISA), radioimmunoassays (RIA), immunoradiometric assays (IRMA), fluorescence resonance energy transfer (FRET) or time-resolved FRET, and immunoenzymatic assays (IEMA), including sandwich assays using monoclonal and/or polyclonal antibodies.

For example, binding between ZCCHC and LIN28 polypeptides may be studied in vitro by labelling one with a detectable label and bringing it into contact with the other which has been immobilised on a solid support. This may be performed in the presence of a test compound.

Suitable detectable labels, especially for peptidyl substances include ³⁵S-methionine, which may be incorporated into recombinantly produced peptides and polypeptides. Recombinantly produced peptides and polypeptides may also be expressed as fusion proteins containing a label, for example a fluorescent label, such as GFP or mCherry, or an epitope which can be labelled with an antibody.

In a scintillation proximity assay, a biotinylated protein fragment may be bound to streptavidin coated scintillant-impregnated beads (for example, produced by Amersham). Binding of radiolabelled peptide is then measured by determination of radioactivity-induced scintillation as the radioactive peptide binds to the immobilized fragment. Agents that block this binding are inhibitors of the interaction.

A polypeptide may be immobilized using an antibody against that polypeptide which is bound to a solid support or via other technologies which are known per se. A preferred in vitro interaction may utilise a fusion protein including glutathione-S-transferase (GST). This may be immobilized on glutathione agarose beads. In an in vitro format, a test compound can be assayed by determining its ability to diminish the amount of labelled peptide or polypeptide which binds to the immobilized GST-fusion polypeptide. This may be determined by fractionating the glutathione-agarose beads by SDS-polyacrylamide gel electrophoresis.

Alternatively, the beads may be rinsed to remove unbound protein and the amount of bound protein determined by counting the amount of label present, for example, using a suitable scintillation counter.

Of course, the person skilled in the art will design any appropriate control experiments with which to compare results obtained in methods of the invention.

Methods described herein may also take the form of in vivo methods. In vivo methods may be performed in a cell line such as a yeast strain, insect or mammalian cell line, for example CHO, HeLa or COS cells, in which the relevant polypeptides or peptides are expressed from one or more vectors introduced into the cell.

Suitable techniques include the yeast two-hybrid system^{61,62}. This system may be used to screen for compounds able to disrupt binding between LIN28 and ZCCHC polypeptides. For instance, the polypeptides may be expressed in a yeast two-hybrid system (e.g. one as a GAL4 DNA binding domain fusion, the other as a GAL4 activator fusion) which is treated with test substances. The absence of the end-point which normally indicates interaction between the pathway components (e.g. the absence of a blue colour generated by β-galactosidase), when a test compound is applied, indicates that the compound disrupts interaction between the two components, and may therefore modulate miRNA activity as described herein.

ZCCHC polypeptides suitable for use in the methods described herein include any eukaryotic ZCCHC polypeptide, such as C. elegans PUP2, in particular a mammalian ZCCHC polypeptide, such as a human ZCCHC polypeptide. Suitable ZCCHC polypeptides possess uridylyl-transferase activity.

C. elegans PUP2 (GeneID: 175708) may have the amino acid sequence of NP_{—}498100.1 GI: 17554126 and be encoded by NM_{—}065699.2 GI: 71988416.

A human ZCCHC polypeptide may be a ZCCHC11 polypeptide or a ZCCHC6 polypeptide. A ZCCHC11 polypeptide (also known as PAPD3; Gene ID: 23318) may have the amino acid sequence of any one of SEQ ID NOS: 3 to 8 or may be an allele, homologue or variant of any one of these sequences.

For example a human ZCCHC11 polypeptide may have the amino acid sequence of NP_{—}001009881.1 GI:57863248 (isoform a) and be encoded by NM_{—}001009881.1 GI:57863247; the amino acid sequence of NP_{—}001009882.1 GI:57863250 (isoform c) and be encoded by NM_{—}001009882.1 GI:57863249; or the amino acid sequence of NP_{—}056084.1 GI:57863246 (isoform b) and be encoded by NM_{—}015269.1 GI:57863245.

A ZCCHC6 polypeptide (also known as PAPD6; Gene ID: 79670) may have the amino acid sequence of any one of SEQ ID NOS: 10 to 19 or may be an allele, homologue or variant of any one of these sequences.

For example a human ZCCHC6 polypeptide may have the amino acid sequence of NP_{—}078893.2 GI: 58331272 and be encoded by the nucleic acid sequence of NM_{—}024617.2 GI:58331271.

The sequences of numerous eukaryotic ZCCHC polypeptides are publically available from sequence databases (e.g. Genbank).

A ZCCHC polypeptide may, for example, comprise or consist of the amino acid sequence of SEQ ID NOS: 2, 3 or 9 or may be an allele, homologue or variant of any one of these sequences.

An allele, homologue or variant of a wild-type ZCCHC sequence, for example SEQ ID NOs: SEQ ID NOS: 2, 3 or 9, may differ from the wild-type sequence by the addition, deletion, substitution and/or insertion of one or more amino acids, provided uridylyl-transferase activity or LIN28-dependent RNA polyuridylase activity is retained. For example, a sequence variant, homologue or allele may differ from the reference ZCCHC amino acid sequence described herein (e.g SEQ ID NOS: 2, 3 or 9) by addition, deletion or substitution of 1 or more amino acids, for example, up to 5 amino acids, up to 10 amino acids, up to 20 amino acids, up to 30 amino acids, up to 40 amino acids, up to 50 amino acids or up to 100 amino acids.

A suitable LIN28 polypeptide may include any eukaryotic LIN28, such as C. elegans LIN28, or a mammalian LIN28 such as human LIN28. The sequences of various eukaryotic LIN28 polypeptides are publically available from sequences databases.

For example, C. elegans LIN28 (GeneID: 172626) may have the amino acid sequence of NP_{—}001021085.1 GI: 71983217 and be encoded by NM_{—}001025914.3 GI: 193202517, and human LIN28 (GeneID: 79727) may have the amino acid sequence of NP_{—}078950,1 GI: 13375938 and be encoded by the nucleotide sequence of NM_{—}024674.4 GI: 94536796.

A LIN28 polypeptide may for example comprise or consist of the amino acid sequence of one of SEQ ID NOs: 20 or 22 or may be an allele, homologue or variant of any one of these sequences.

An allele or variant of a wild-type LIN28 sequence, for example SEQ ID NOs: 20 or 22, may differ from the wild-type sequence by the addition, deletion, substitution and/or insertion of one or more amino acids, provided the activity of pre-miRNA and ZCCHC poly(U) polymerase binding is retained. For example, a sequence variant, homologue or allele may differ from the reference LIN28 amino acid sequence described herein (e.g SEQ ID Nos: 20 or 22) by addition, deletion or substitution of 1 or more amino acids, for example, up to 5 amino acids, up to 10 amino acids, up to 20 amino acids, up to 30 amino acids, up to 40 amino acids, up to 50 amino acids or up to 100 amino acids. Suitable variants, homologues or alleles of Lin28 may comprise a cold-shock domain.

An allele or variant of a reference LIN28 or ZCCHC amino acid sequence described herein (e.g SEQ ID NOs: 2, 3, 9, 20 or 22) may comprise an amino acid sequence which shares greater than 30% sequence identity with the wild-type sequence, greater than 40%, greater than 50%, greater than 60%, greater than 70%, greater than 80%, greater than 90%, greater than 95% or greater than 98%.

Sequence identity is commonly defined with reference to the algorithm GAP (Genetics Computer Group, Madison, Wis.). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST⁶³, FASTA⁶⁴, or the Smith-Waterman algorithm⁶⁵, or the TBLASTN program⁶³, generally employing default parameters. In particular, the psi-Blast algorithm may be used⁶⁶. Sequence identity similarity may also be determined using Genomequest™ software (Gene-IT, Worcester Mass. USA).

Sequence comparisons are preferably made over the full-length of the relevant sequence described herein.

ZCCHC and LIN28 polypeptides may include fragments of the full-length ZCCHC and LIN28 polypeptide sequences which retain all or part of the activity of the full-length protein. Suitable fragments may be generated and used in the methods described herein, whether in vitro or in vivo.

Suitable ways of generating fragments include, but are not limited to, recombinant expression of a fragment from encoding DNA. For example, fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Small fragments (e.g. up to about 20 or 30 amino acids) may also be generated using peptide synthesis methods which are well known in the art as further described below.

A fragment of a full-length sequence may consist of fewer amino acids than the full-length sequence. For example a fragment may consist of at least 80, at least 90 or at least 100 amino acids of the full length sequence but 800 or less, 700 or less, 600 or less, 500 or less, 250 or less, 200 or less, 150 or less, or 125 or less amino acids of the full length sequence.

Methods described herein may be in vivo cell-based methods, or in vitro non-cell-based methods. The precise format for performing methods of the invention may be varied by those of skill in the art using routine skill and knowledge.

Test compounds for use in methods of the invention may be natural or synthetic chemical compounds used in drug screening programmes and may include, for example, small organic molecules; polypeptides, such as antibodies and antibody fragments; and nucleic acids, such as aptamers. Extracts of plants that contain several characterised or uncharacterised components may also be used.

Combinatorial library technology provides an efficient way of testing a potentially vast number of different compounds for ability to modulate an interaction. Such libraries and their use are known in the art, for all manner of natural products, small molecules and peptides, among others. The use of peptide libraries may be preferred in certain circumstances.

The amount of test substance or compound which may be employed in a method described herein will normally be determined by trial and error depending upon the type of compound used. Typically, from about 0.1 to 100 μM concentrations of putative inhibitor compound may be used, for example from 1 to 10 μM. When cell-based methods are employed, the test substance or compound is desirably membrane permeable in order to access the interacting polypeptides.

One class of putative agents for modulating miRNA activity can be derived from the ZCCHC and LIN28 polypeptides as described above. Membrane permeable peptide fragments of from 5 to 40 amino acids, for example, from 6 to 10 amino acids may be tested for their ability to disrupt, for example, the ZCCHC/LIN28 interaction.

The inhibitory properties of a peptide fragment as described above may be increased by the addition of one of the following groups to the C terminal: chloromethyl ketone, aldehyde and boronic acid. These groups are transition state analogues for serine, cysteine and threonine proteases. The N terminus of a peptide fragment may be blocked with carbobenzyl to inhibit aminopeptidases and improve stability (Proteolytic Enzymes 2nd Ed, Edited by R. Beynon and J. Bond, Oxford University Press, 2001).

Antibodies, antibody fragments and antibody derivatives and non-immunoglobulin binding molecules, such as aptamers, trinectins, anticalins, kunitz domains, transferrins, nurse shark antigen receptors and sea lamprey leucine-rich repeat proteins, directed to the active site of ZCCHC poly(U)polymerase or the site of interaction between the ZCCHC poly(U)polymerase and LIN28 form a further class of putative agents for modulating the miRNA activity. For example, a suitable antibody may bind an epitope within ZCCHC poly(U)polymerase or LIN28. Candidate inhibitor antibody molecules may be characterised and their binding regions determined to provide single chain antibodies or fragments thereof which are responsible for disrupting the interaction. Suitable antibodies may be obtained using techniques which are standard in the art, including, for example immunising a mammal with a suitable peptide, such as a fragment of Lin28 or ZCCHC poly(U)polymerase, or isolating a specific antibody from a recombinantly produced library of expressed immunoglobulin variable domains, e.g. using lambda bacteriophage or filamentous bacteriophage which display functional immunoglobulin binding domains on their surfaces; for instance see WO92/01047.

Other candidate compounds for modulating miRNA activity may be based on modelling the 3-dimensional structure of LIN28 and ZCCHC, either alone or in combination, and using rational drug design to provide candidate compounds with particular molecular shape, size and charge characteristics. For example, a chemical compound may be modelled to resemble the three dimensional structure of the component in an area which contacts another component, and in particular the arrangement of the key amino acid residues as they appear. Techniques for the rational design of compounds that bind to target proteins are well-known in the art.

Firstly, the particular parts of a compound that are critical and/or important in modulating the interaction of Lin28 and ZCCHC poly(U)polymerase are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR.

Computational analysis, similarity mapping (which models the charge and/or volume of a pharmacophore, rather than the bonding between atoms) and other techniques can be used in this modelling process.

In a variant of this approach, the three-dimensional structure of LIN28 and ZCCHC poly(U)polymerase are modelled. This allows the model to take account of changes conformation on binding in the optimisation of the lead compound.

A template molecule is then selected onto which chemical groups which mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. The modified compounds found by this approach can then be screened to see whether they have the target property, or to what extent they exhibit it. Modified compounds include mimetics of the lead compound.

Another class of suitable ZCCHC poly(U) polymerase modulators inhibitors includes nucleic acid encoding part or all of the ZCCHC amino acid sequence, or the complement thereof, which inhibit activity or function by down-regulating production of active ZCCHC polypeptide. For instance, expression of a ZCCHC polypeptide may be inhibited using anti-sense or RNAi technology. The use of these approaches to down-regulate gene expression is now well-established in the art.

Anti-sense oligonucleotides may be designed to hybridise to the complementary sequence of nucleic acid, pre-mRNA or mature mRNA, interfering with the production of the base excision repair pathway component so that its expression is reduced or completely or substantially completely prevented. In addition to targeting coding sequence, anti-sense techniques may be used to target control sequences of a gene, e.g. in the 5′ flanking sequence, whereby the anti-sense oligonucleotides can interfere with expression control sequences. The construction of anti-sense sequences and their use is described for example by Peyman and Ulman and Crooke^{67,68}.

Oligonucleotides may be generated in vitro or ex vivo for administration or anti-sense RNA may be generated in vivo within cells in which down-regulation is desired. Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g. about 15, 16 or 17.

An alternative to anti-sense is to use a copy of all or part of the target gene inserted in sense, that is the same, orientation as the target gene, to achieve reduction in expression of the target gene by co-suppression^{73,74}. Double stranded RNA (dsRNA) has been found to be even more effective in gene silencing than both sense or antisense strands alone⁷⁵. dsRNA mediated silencing is gene specific and is often termed RNA interference (RNAi).

RNA interference is a two-step process. First, dsRNA is cleaved within the cell to yield short interfering RNAs (siRNAs) of about 21-23 nt length with 5′ terminal phosphate and 3′ short overhangs (˜2 nt). The siRNAs target the corresponding mRNA sequence specifically for destruction⁷⁶. RNAi may also be efficiently induced using chemically synthesized siRNA duplexes of the same structure with 3′-overhang ends⁶⁹. Synthetic siRNA duplexes have been shown to specifically suppress expression of endogenous and heterologeous genes in a wide range of mammalian cell lines⁷⁰.

Another possibility is that nucleic acid is used which on transcription produces a ribozyme, able to cut nucleic acid at a specific site—thus also useful in influencing gene expression. Background references for ribozymes include Kashani-Sabet and Scanlon⁷¹, and Mercola and Cohen⁷².

Further optimisation or modification can then be carried out to arrive at one or more final compounds for further testing, for example in vitro, in vivo or clinical testing.

Methods as described herein may comprise the step of identifying a test compound which alters or modulates (e.g. increases or decreases) the binding of the ZCCHC polypeptide to the LIN28 polypeptide and/or the polyuridylation activity of the ZCCHC polypeptide as a candidate modulator of let-7 activity.

A test compound which alters the binding of the ZCCHC polypeptide to the LIN28 polypeptide and/or the polyuridylation activity of the ZCCHC polypeptide is a candidate modulator of miRNA activity.

A compound which increases ZCCHC binding to LIN28 and/or ZCCHC polyuridylase activity may be identified as a candidate inhibitor of miRNA activity. A compound which decreases or reduces ZCCHC binding to LIN28 and/or ZCCHC polyuridylase activity may be identified as a candidate enhancer of miRNA activity.

Following identification of a compound which modulates miRNA activity, a method may further comprise modifying the compound to optimise its pharmaceutical properties. This may be done by modelling techniques as described above.

A test compound identified using one or more initial screens as having ability to modulate e.g. increase or decrease ZCCHC binding to LIN28 and/or ZCCHC polyuridylase activity and thereby modulate miRNA activity, may be assessed further using one or more secondary screens.

Let-7 regulates the expression of the K-ras and HMGA2 proto-oncogenes. A reporter gene such as luciferase or GFP may be linked to K-ras or HMGA2 regulatory sequences and the expression of the reporter in the presence and absence of the test compound determined. The expression of the reporter gene is indicative of the activity of let-7 and alterations in expression in the presence relative to the absence of the test compound are indicative that the compound modulates let-7 activity.

The effect of the test compound may be determined using a qRT-PCR analysis of a target gene for mature miRNA. For example, the effect of the test compound on the expression of the K-ras and HMGA2 proto-oncogenes may be determined.

A secondary screen may involve testing for a biological function or activity in vitro and/or in vivo, e.g. in an animal model. For example, the effect of the test compound on one or more of; let-7 levels in cancer cell lines, proliferation and differentiation of cancer stem populations; and let-7 tumour mouse models may be determined.

The ability of a test compound to modulate cell development or differentiation may be determined, for example in a tissue culture assay with a suitable cell line.

For example, the effect of the compound on the differentiation and fusion of lateral seam cells into syncytium may be determined in C. elegans. Defective fusion of lateral seam cells is indicative of in vivo inhibition of ZCCHC binding to LIN28 and/or ZCCHC polyuridylase activity.

Following identification of a test compound which modulates miRNA activity, the compound may be isolated and/or purified or alternatively it may be synthesised using conventional techniques of recombinant expression or chemical synthesis. Furthermore, it may be manufactured and/or used in preparation, i.e. manufacture or formulation, of a composition such as a medicament, pharmaceutical composition or drug. These may be administered to individuals for the treatment of a disease or medical condition.

Compounds identified as candidate modulators of miRNA activity using any of the methods described herein may be useful in modulating (i.e. increasing or decreasing) cell development and or differentiation in a therapeutic context. For example, a compound identified as a candidate modulator of miRNA activity using any of the methods described herein may also be useful the treatment of neurodegenerative diseases, such as Alzheimer's disease and Parkinson's disease, liver dysfunctions, hepatic viral infections such as HCV, myopathies, heart disease, for example sustained cardiac hypertrophy and arrthymogenesis, diabetes and cancer, including leukaemia.

In some embodiments, a candidate modulator of let-7 may be identified.

A compound identified as a candidate inhibitor of let-7 activity using any of the methods described herein may be useful in inducing, stimulating or maintaining pluripotency in a cell. A compound identified as a candidate promoter of let-7 activity using any of the methods described herein may be useful in reducing proliferation or increasing differentiation. This may be useful, for example, in the treatment of cancer or other proliferative conditions⁵⁰⁻⁵³.

A cancer may include any type of solid cancer or malignant lymphoma and especially leukaemia, sarcomas, skin cancer, bladder cancer, breast cancer, uterus cancer, ovary cancer, prostate cancer, lung cancer, colorectal cancer, cervical cancer, liver cancer, head and neck cancer, oesophageal cancer, pancreas cancer, renal cancer, stomach cancer and cerebral cancer. In some preferred embodiments, the cancer condition may be lung cancer, liver cancer, melanoma, ovarian cancer, or colon cancer.

A compound identified as a candidate promoter of let-7 activity using any of the methods described herein may also be useful the treatment of coronary heart disease and diabetes.

Examples of compounds for use in treating diseases by the modulation of the activity of a ZCCHC poly(U) polymerase and/or by the alteration of let-7 expression are described in the Compounds section below.

### Compounds

The present inventors also provide compounds for use in treating diseases by the modulation of the activity of a ZCCHC poly(U) polymerase and/or by the alteration of let-7 expression. The identification of such compounds is described above.

MicroRNAs (miRNAs) are small noncoding RNAs, ˜22 nucleotides in length, that repress target messenger RNAs (mRNAs) through an antisense mechanism. The let-7 miRNA was originally discovered in the nematode Caenorhabditis elegans, where it regulates cell proliferation and differentiation, but subsequent work has shown that both its sequence and its function are highly conserved in mammals. Recent results have now linked decreased let-7 expression to increased tumorigenicity and poor patient prognosis.

In review articles by Büssing⁵⁴ and Schickel⁵⁵ a number of disclosures disclosing a possible role of this miRNA in human diseases such as cancer are identified. In particular, Let-7 is downregulated in a number of human cancers such as lung, colon, or ovarian cancer, and it serves as a prognostic marker for disease outcome.

let-7 has been shown to target the oncogenes RAS, MYC, and HMAG2. HMGA2, which is not expressed in most adult tissues, is upregulated in various cancers, such as neuroblastoma, pancreatic cancer, thyroid neoplasms, squamous carcinoma and lung cancer.

Restoration of let-7 expression in tumours by a direct therapeutic approach such as the application of exogenous let-7 RNA run into the obstacle of safe and efficient delivery of RNAs to the target tissue. Therefore, alternative approaches are required.

It is known that Lin28 is a conserved RNA-binding protein, which in mammals controls stem cell lineages and inhibits let-7 miRNA processing in vivo⁶⁻¹⁰.

The present inventors have discovered that Lin-28 and a poly(U) polymerase, ZCCHC, regulate let-7 processing, and therefore by altering the activity of the ZCCHC poly(U) polymerase, let-7 processing can be affected and the diseases discussed above can be treated.

ZCCHC poly(U) polymerases relevant to the present invention may include any eukaryotic ZCCHC polypeptide, such as C. elegans PUP2, in particular a mammalian ZCCHC polypeptide, such as a human ZCCHC polypeptide. Suitable ZCCHC polypeptides possess uridylyl-transferase activity.

A human ZCCHC polypeptide may be a ZCCHC11 polypeptide or a ZCCHC6 polypeptide, or an allele, homologue or variant of these.

Detailed information on the linkage between the modulation of ZCCHC activity and let-7 is discussed above.

Modulation of the activity of a ZCCHC poly(U) polymerase may also alter the expression of other miRNAs. Modulating miRNA activity may treat neurodegenerative diseases, such as Alzheimer's disease and Parkinson's disease, liver dysfunctions, hepatic viral infections such as HCV, myopathies, heart disease, for example sustained cardiac hypertrophy and arrthymogenesis, diabetes and cancer, including leukaemia⁵³.

Therefore, in another aspect the present invention provides compounds which can be used to modulate the activity of a ZCCHC poly(U) polymerase and thus treat diseases ameliorated by the alteration of let-7 expression.

In one aspect the invention provides compounds of formula I:

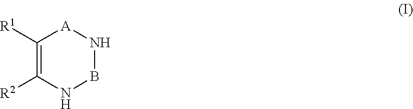

(and isomers, salts, solvates, protected forms, and prodrugs thereof)
for use in treating a disease ameliorated by the alteration of let-7 expression
wherein:
A and B are selected from:
   (i) CH₂ and C═X, where X is O or S;
   (ii) C═X and CH₂; and
   (iii) C═X and C═X;
one of R¹ and R² is H, and the other of R¹ and R² is R, where R is selected from halo, halo-C₁₋₄ alkyl, C₅₋₇ aryl, and C₃₋₇ heterocyclyl.

The first aspect also provides the use of compounds of formula I in the manufacture of a medicament for the treatment of a disease ameliorated by the alteration of let-7 expression and a method of treating a disease ameliorated by the alteration of let-7 expression comprising administering to a patient a compound of formula I.

A further aspect of the invention provides compounds of formula IIa or IIb:

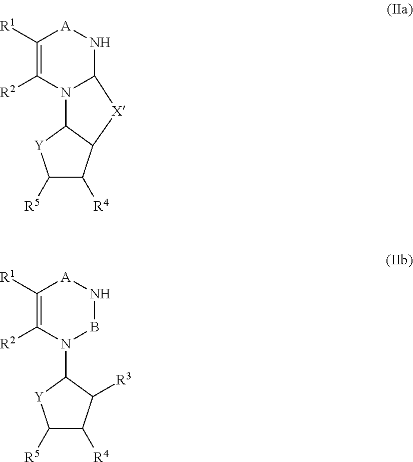

(and isomers, salts, solvates, protected forms, and prodrugs thereof)
for use in treating diseases ameliorated by the alteration of let-7 expression
wherein:
in formula IIa A is CH₂ or C═X, where X is O or S, and X′ is O or S;
in formula IIb, A and B are selected from:
   (i) CH₂ and C═X, where X is O or S;
   (ii) C═X and CH₂; and
   (iii) C═X and C═X;
Y is selected from O, S and NH;
R¹ and R² are independently selected from H and R, where R is selected from halo, halo-C₁₋₄ alkyl, C₅₋₄ aryl, and C₃₋₄ heterocyclyl;
R³ (where present) is selected from H, OH, N₃ and R;
R⁴ is selected from H, OH, N₃ and R; and
R⁵ is selected from CH₂OH, and a group of formula IIIa or IIIb:

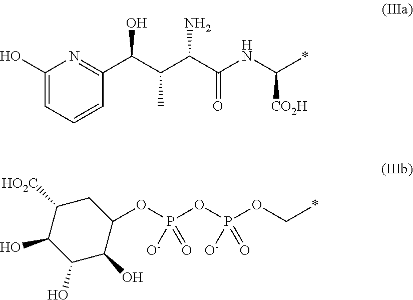

Also provided is the use of compounds of formula IIa or IIb in the manufacture of a medicament for the treatment of a disease ameliorated by the alteration of let-7 expression and a method of treating a disease ameliorated by the alteration of let-7 expression comprising administering to a patient a compound of formula IIa or IIb.

Diseases ameliorated by the alteration of let-7 expression include, but are not limited to cancer or other proliferative conditions, coronary heart disease and diabetes. A cancer may include any type of solid cancer or malignant lymphoma and especially leukaemia, sarcomas, skin cancer, bladder cancer, breast cancer, uterus cancer, ovary cancer, prostate cancer, lung cancer, colorectal cancer, cervical cancer, liver cancer, head and neck cancer, oesophageal cancer, pancreas cancer, renal cancer, stomach cancer and cerebral cancer. In some preferred embodiments, the cancer condition may be lung cancer, liver cancer, melanoma, ovarian cancer, or colon cancer.

As mentioned above, modulation of the activity of a ZCCHC polypeptide may also alter the expression of other miRNAs. Accordingly, further aspects of the invention include:

(a) compounds of formula I, IIa or IIb (and isomers, salts, solvates, protected forms, and prodrugs thereof) for use in treating a disease ameliorated by the modulation of ZCCHC polypeptide activity;
(b) use of compounds of formula I, IIa or IIb in the manufacture of a medicament for the treatment of a disease ameliorated by the modulation of ZCCHC polypeptide activity; and
(c) a method of treating a disease ameliorated by the alteration of ZCCHC polypeptide activity comprising administering to a patient a compound of formula I, IIa or IIb.

Diseases ameliorated by the alteration of ZCCHC polypeptide activity include, but are not limited to, diseases ameliorated by the alteration of let-7 expression as described above and neurodegenerative diseases, such as Alzheimer's disease and Parkinson's disease, liver dysfunctions, hepatic viral infections such as HCV, myopathies, heart disease, for example sustained cardiac hypertrophy and arrthymogenesis and diabetes.

### DEFINITIONS

Halo: —F, —Cl, —Br, and —I.

C₁₋₄ alkyl: The term “C₁₋₄ alkyl” as used herein, pertains to a monovalent moiety obtained by removing a hydrogen atom from a carbon atom of an aliphatic hydrocarbon compound having from 1 to 4 carbon atoms, and which may be saturated or unsaturated (e.g. partially unsaturated, fully unsaturated). Thus, the term “C₁₋₄ alkyl” includes the sub-classes “C₂₋₄ alkenyl” and “C₂₋₄ alkynyl”.

In the context of alkyl groups, the prefixes (e.g. C₁₋₄) denote the number of carbon atoms, or range of number of carbon atoms. For example, the term “C₁₋₄ alkyl”, as used herein, pertains to an alkyl group having from 1 to 4 carbon atoms. Note that the first prefix may vary according to other limitations; for example, for unsaturated alkyl groups, the first prefix must be at least 2.

Examples of saturated C₁₋₄ alkyl groups are methyl (C₁), ethyl (C₂), propyl (C₃) and butyl (C₄).

Examples of saturated C₁₋₄ linear alkyl groups are methyl (C₁), ethyl (C₂), n-propyl (C₃) and n-butyl (C₄).

Examples of saturated C₁₋₄ branched alkyl groups include iso-propyl (C₃), iso-butyl (C₄), sec-butyl (C₄) and tert-butyl (C₄).

Alkenyl: The term “C₂₋₄ alkenyl”, as used herein, pertains to an alkyl group having one or more carbon-carbon double bonds. Examples of alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂) and butenyl (C₄).

Alkynyl: The term “C₂₋₄ alkynyl”, as used herein, pertains to an alkyl group having one or more carbon-carbon triple bonds. Examples of alkynyl groups include, but are not limited to, ethynyl (ethinyl, —C≡CH) and 2-propynyl (propargyl, —CH₂—C≡CH).

Halo-C₁₋₄ alkyl: The term “halo-C₁₋₄ alkyl” as used herein, pertains to C₁₋₄ alkyl group, as defined above, bearing one or more halo substituents. The alkyl group may have one or more substituents, for example, two or three. Groups where all the hydrogens are substituted by halo groups are perhalo-C₁₋₄ alkyl groups.

C₅₋₇ aryl: The term “C₅₋₇ aryl” as used herein, pertains to a monovalent moiety obtained by removing a hydrogen atom from an aromatic ring atom of a C₅₋₇ aromatic compound, said compound having one aromatic ring having from 5 to 7 ring atoms.

The ring atoms may be all carbon atoms, as in “carboaryl groups” in which case the group may conveniently be referred to as a “C₅₋₇ carboaryl” group.

Examples of C₅₋₇ aryl groups which do not have ring heteroatoms (i.e. C₅₋₇ carboaryl groups) include, but are not limited to, those derived from benzene (i.e. phenyl) (C₆).

Alternatively, the ring atoms may include one or more heteroatoms, including but not limited to oxygen, nitrogen, and sulfur, as in “heteroaryl groups”. In this case, the group may conveniently be referred to as a “C₅₋₇ heteroaryl” group, wherein “C₅₋₇” denotes ring atoms, whether carbon atoms or heteroatoms. The ring may preferably have from 1 to 4 ring heteroatoms.

Examples of C₅₋₇ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine) and triazine.

The heteroaryl group may be bonded via a carbon or hetero ring atom.

The C₅₋₇ aryl group may bear one or more halo substituents.

C₃₋₇ heterocyclyl: The term “C₃₋₇ heterocyclyl”, as used herein, pertains to a monovalent moiety obtained by removing a hydrogen atom from a ring atom of a heterocyclic compound, which moiety has from 3 to 7 ring atoms (unless otherwise specified), of which from 1 to 4 are ring heteroatoms.

In this context, the prefixes (e.g. C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

The C₃₋₇ heterocyclyl group may bear one or more halo substituents.

### Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and L-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

If the compound is in crystalline form, it may exist in a number of different polymorphic forms.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic and salt forms thereof, for example as discussed below.

Unless otherwise specified, a reference to a particular compound also includes solvates thereof, for example as discussed below.

Unless otherwise specified, a reference to a particular compound also includes prodrugs thereof, for example as discussed below.

Unless otherwise specified, a reference to a particular compound also includes protected forms thereof, for example as discussed below.

Unless otherwise specified, a reference to a particular compound also includes different polymorphic forms thereof, for example as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al.⁵⁶.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, toluenesulfonic, methanesulfonic, ethanesulfonic, ethane disulfonic, oxalic, isethionic, valeric, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis⁵⁷.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO.).

For example, a carboxylic acid group may be protected as an ester for example, as: an C₁₋₇ alkyl ester (e.g. a methyl ester; a t-butyl ester); a C₁₋₇ haloalkyl ester (e.g. a C₁₋₇ trihaloalkyl ester); a triC₁₋₇ alkylsilyl-C₁₋₇ alkyl ester; or a C₅₋₂₀ aryl-C₁₋₇ alkyl ester (e.g, a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₂₀ alkyl (e.g. -Me, -Et); C₁₋₇ aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy)carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Further suitable prodrug forms include phosphonate and glycolate salts. In particular, hydroxy groups (—OH), can be made into phosphonate prodrugs by reaction with chlorodibenzylphosphite, followed by hydrogenation, to form a phosphonate group —O—P(═O)(OH)₂. Such a group can be cleared by phosphotase enzymes during metabolism to yield the active drug with the hydroxy group.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### Further Embodiments

The limitations and embodiments discussed below may be combined in any manner possible.

### Formula I

### A & B

In some embodiments, both A and B are C═X. In other embodiments, only one of A and B are C═X.

In some of these embodiments, X is O. In others of these embodiments, X is S.

It may be preferred that both A and B are C═O.

### R¹ and R²

In some embodiments, R¹ is H and R² is R

In other embodiments R² is H and R¹ is R.

If R is halo, then the halo group may be I, Br, Cl and F. In some embodiments, the halo group is selected from Br, Cl and F or Cl and F. In certain embodiments, the halo group is F.

If R is halo-C₁₋₄ alkyl, in some embodiments, the C₁₋₄ alkyl group is a C₁₋₃ (e.g. methyl, ethyl, propyl, ethylene, propylene) or C₁₋₂ alkyl group (e.g methyl, ethyl, ethylene). In some embodiments, it is a C₁ alkyl group (i.e. methyl).

If R is halo-C₁₋₄ alkyl, in some embodiments, there may be one, two, three or more halo substituent groups. In some embodiments, all the hydrogen atoms are replaced by halo groups.

Particular halo-C₁₋₄ alkyl groups of interest include, bromoethylene (BrCH═CH—), chloromethyl (ClH₂C—) and trifluoromethyl (F₃C—).

If R is C₅₋₇ aryl, in some embodiments R is C₅₋₆ aryl. In particular, R may be phenyl (C₆ carboaryl) or C₅₋₆ heteroaryl where there is a single ring heteroatom, e.g. furanyl, thienyl, pyridyl.

If R is C₅₋₇ aryl, in some embodiments the C₅₋₇ aryl bears one or more halo substituents, for example 1, 2 or 3 halo substituents.

If R is C₃₋₇ heterocyclyl, in some embodiments R is C₆₋₇ heterocyclyl. In some of these embodiments, there is only a single ring heteroatom.

If R is C₃₋₇ heterocyclyl, in some embodiments the C₅₋₇ aryl bears one or more halo substituents, for example 1, 2 or 3 halo substituents.

### Formula Ix

In some embodiments, the compound compounds of formula I may be of formula Ix:

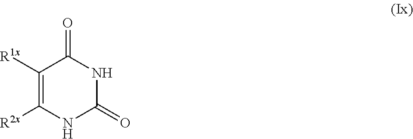

wherein one of R^{1x} and R^{2x} is H, and the other of R¹ and R² is selected from halo (e.g. Br, Cl, F) and halo-C₁₋₄ alkyl.

### Embodiments of Formula I

Embodiments of the compounds of formula (I) include, but are not limited to:

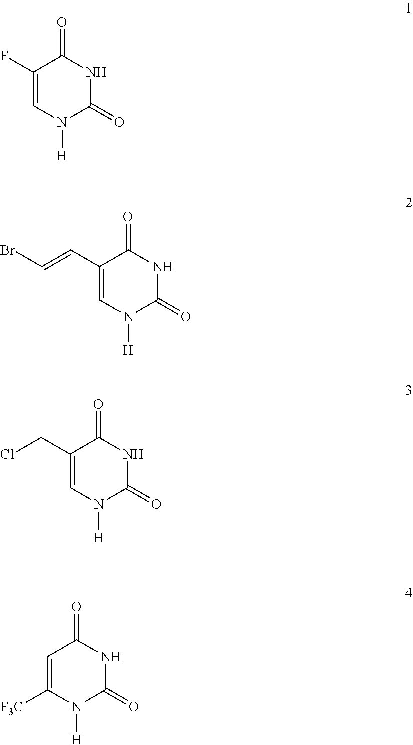

### Formula II

### A and X′—Formula IIa

In some embodiments, A can be O or S. It may be preferred that A is O.

In some embodiments, X′ is O or S. It may be preferred that X′ is O.

### A & B—Formula IIb

In some embodiments, both A and B are C═X. In other embodiments, only one of A and B are C═X.

In some of these embodiments, X is O. In others of these embodiments, X is S.

It may be preferred that both A and B are C═O.

### Y

Y may be O, S and NH. In some embodiments, Y is O or S. In particular embodiments, Y is O.

### R¹ and R²

In some embodiments, R¹ is H and R² is R

In other embodiments R² is H and R¹ is R.

In some embodiments, R¹ and R² are both H, and this may be preferred.

If R is halo, then the halo group may be I, Br, Cl and F. In some embodiments, the halo group is selected from Br, Cl and F or Cl and F. In certain embodiments, the halo group is F.

If R is halo-C₁₋₄ alkyl, in some embodiments, the C₁₋₄ alkyl group is a C₁₋₃ (e.g. methyl, ethyl, propyl, ethylene, propylene) or C₁₋₂ alkyl group (e.g methyl, ethyl, ethylene). In some embodiments, it is a C₁ alkyl group (i.e. methyl).

If R is halo-C₁₋₄ alkyl, in some embodiments, there may be one, two, three or more halo substituent groups. In some embodiments, all the hydrogen atoms are replaced by halo groups.

Particular halo-C₁₋₄ alkyl groups of interest include, bromoethylene (BrCH═CH—), chloromethyl (ClH₂C—) and trifluoromethyl (F₃C—).

If R is C₅₋₇ aryl, in some embodiments R is C₅₋₆ aryl. In particular, R may be phenyl (C₆ carboaryl) or C₅₋₆ heteroaryl where there is a single ring heteroatom, e.g. furanyl, thienyl, pyridyl.

If R is C₆₋₇ aryl, in some embodiments the C₅₋₇ aryl bears one or more halo substituents, for example 1, 2 or 3 halo substituents.

If R is C₃₋₇ heterocyclyl, in some embodiments R is C₅₋₇ heterocyclyl. In some of these embodiments, there is only a single ring heteroatom.

If R is C₃₋₇ heterocyclyl, in some embodiments the C₅₋₇ aryl bears one or more halo substituents, for example 1, 2 or 3 halo substituents.

### R³ and R⁴

In compounds of formula IIa, R⁴ may be H, OH, N3 and R.

In some embodiments R⁴ is H, OH or halo (e.g. F and Cl). It may be H or OH. In some embodiments, R⁴ is OH.

In compounds of formula IIb, R³ is selected from H, OH, N₃ and R, and R⁴ may be H, OH, N₃ and R.

In some embodiments, R³ is selected from OH and N.

In some embodiments, R⁴ is selected from H, OH, F and N₃.

If R is halo, then the halo group may be I, Br, Cl and F. In some embodiments, the halo group is selected from Br, Cl and F or Cl and F. In certain embodiments, the halo group is F.

If R is halo-C₁₋₄ alkyl, in some embodiments, the C₁₋₄ alkyl group is a C₁₋₃ (e.g. methyl, ethyl, propyl, ethylene, propylene) or C₁₋₂ alkyl group (e.g methyl, ethyl, ethylene). In some embodiments, it is a C₁ alkyl group (i.e. methyl).

If R is halo-C₁₋₄ alkyl, in some embodiments, there may be one, two, three or more halo substituent groups. In some embodiments, all the hydrogen atoms are replaced by halo groups.

Particular halo-C₁₋₄ alkyl groups of interest include, bromoethylene (BrCH═CH—), chloromethyl (ClH₂C—) and trifluoromethyl (F₃C—).

If R is C₅₋₇ aryl, in some embodiments R is C₅₋₆ aryl. In particular, R may be phenyl (C₆ carboaryl) or C₅₋₆ heteroaryl where there is a single ring heteroatom, e.g. furanyl, thienyl, pyridyl.

If R is C₅₋₇ aryl, in some embodiments the C₅₋₇ aryl bears one or more halo substituents, for example 1, 2 or 3 halo substituents.

If R is C₃₋₇ heterocyclyl, in some embodiments R is C₅₋₇ heterocyclyl. In some of these embodiments, there is only a single ring heteroatom.

If R is C₃₋₇ heterocyclyl, in some embodiments the C₅₋₇ aryl bears one or more halo substituents, for example 1, 2 or 3 halo substituents.

### R⁵

In some embodiments R⁵ is CH₂OH.

When R⁵ is or formula IIIa or IIIb, it may be preferred that the compound is of formula IIb, and that both A and B are C═O, X is O, and that both R³ and R⁴ are OH.

### Formula IIx

In some embodiments, the compound compounds of formula IIa and IIb may be of formula IIax and IIbx:

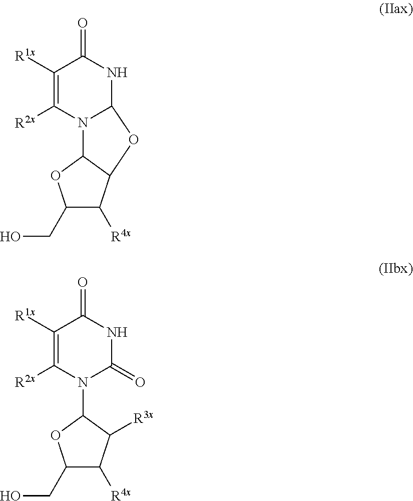

(and isomers, salts, solvates, protected forms, and prodrugs thereof)
for use in treating diseases ameliorated by the alteration of let-7 expression
wherein one of R^{1X} and R^{2X} is H, and the other of R^{1x} and R^{2X} is selected from H, halo and halo-C₁₋₄ alkyl;
R^{3x} (where present) is selected from H, OH, halo and N₃;
R^{4x} is selected from H, OH, halo and N₃.

### Embodiments of Formula II

### Embodiments of the Compounds of Formula (IIa) and (IIb) Include, but are not Limited to:

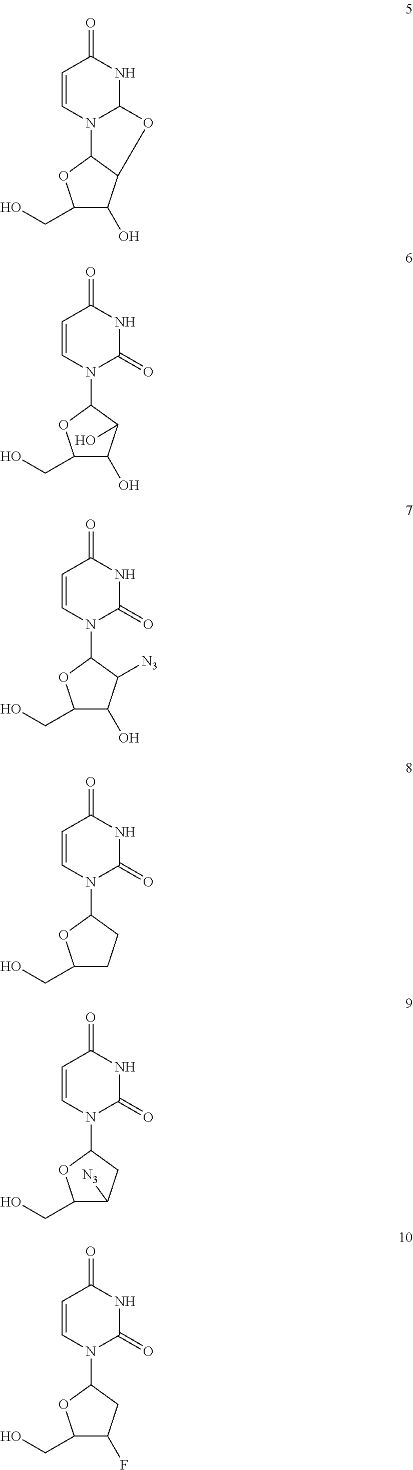

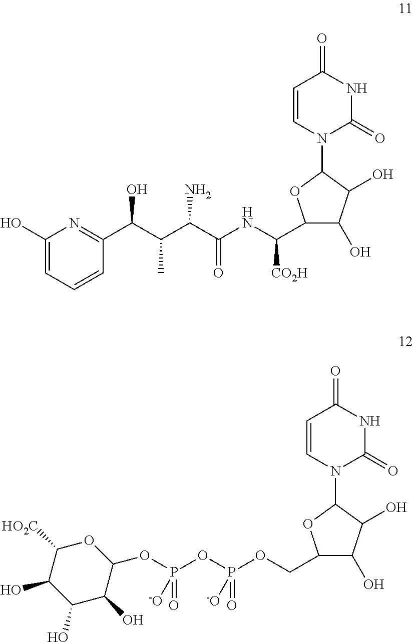

### Synthesis

The compounds of the present invention are commercially available or can be readily synthesised.

### Further Embodiments

The numbered paragraphs below relate to certain embodiments of the invention.

1. A compound of formula I:

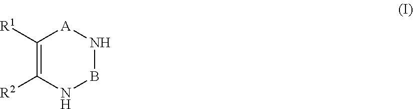

for use in treating a disease ameliorated by the alteration of let-7 expression
wherein:
A and B are selected from:
   (i) CH₂ and C═X, where X is O or S;
   (ii) C═X and CH₂; and
   (iii) C═X and C═X;
one of R¹ and R² is H, and the other of R¹ and R² is R, where R is selected from halo, halo-C₁ alkyl, C₅₋₇ aryl, and C₃₋₇ heterocyclyl.
   2. A compound according to embodiment 1, wherein A and B are both C═O,
   3. A compound according to either embodiment 1 or embodiment 2, wherein R is selected from halo, and halo-C₁₋₄ alkyl.
   4. A compound according to embodiment 3, wherein R is selected from: F, bromoethylene, chloromethyl and trifluoromethyl.
   5. A compound according to embodiment 4, wherein the compound of formula I is:

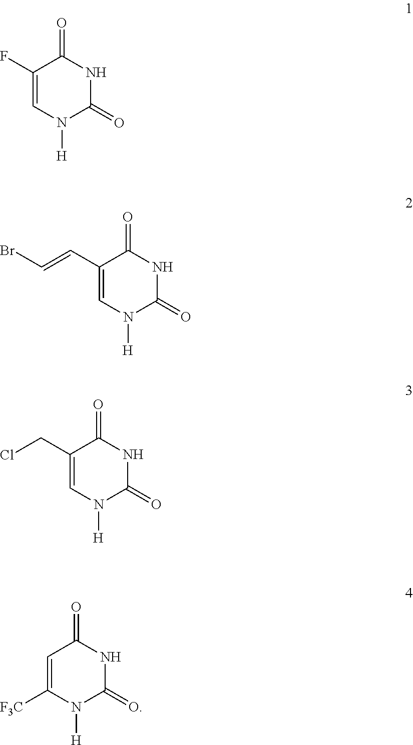

6. A compound of formula IIa or IIb:

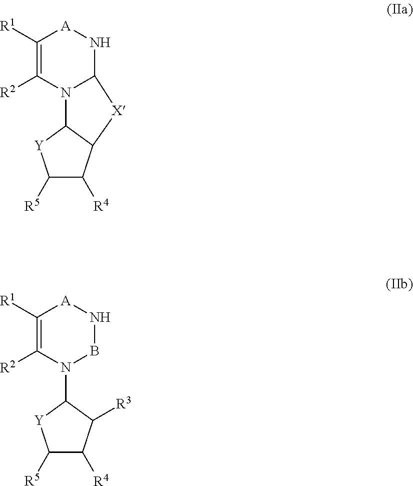

for use in treating diseases ameliorated by the alteration of let-7 expression
wherein:
in formula IIa A is CH₂ or C═X, where X is O or S, and X′ is O or S;
in formula IIb, A and B are selected from:
(i) CH₂ and C═X, where X is O or S;
(ii) C═X and CH₂; and
(iii) C═X and C═X;
Y is selected from O, S and NH;
R¹ and R² are independently selected from H and R, where R is selected from halo, halo-C₁₋₄ alkyl, C₅₋₇ aryl, and C₃₋₇ heterocyclyl;
R³ (where present) is selected from H, OH, N₃ and R;
R⁴ is selected from H, OH, N₃ and R; and
R⁵ is selected from CH₂OH, and a group of formula IIIa or IIIb:

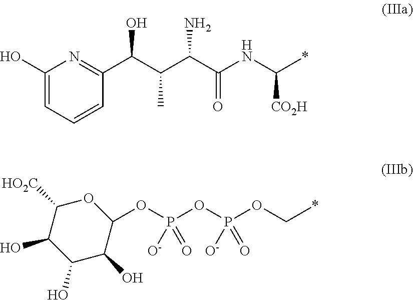

7. A compound according to embodiment 6 of formula IIa, wherein A is O and X′ is O.
8. A compound according to embodiment 6 of formula IIn, wherein A and B are both C═O.
9. A compound according to any one of embodiments 6 to 8, wherein Y is O.
10. A compound according to any one of embodiments 6 to 9, wherein R¹ and R² are H.
11. A compound of formula IIa according to any one of embodiments 6 and 8 to 10, wherein R³ is selected from OH and N₃.
12. A compound according to any one of embodiments 6 to 11, wherein R⁴ is selected from H, OH, F and N₃.
13. A compound according to any one of embodiments 6 to 12, wherein R⁵ is CH₂OH.
14. A compound according to embodiment 6, which is selected from:

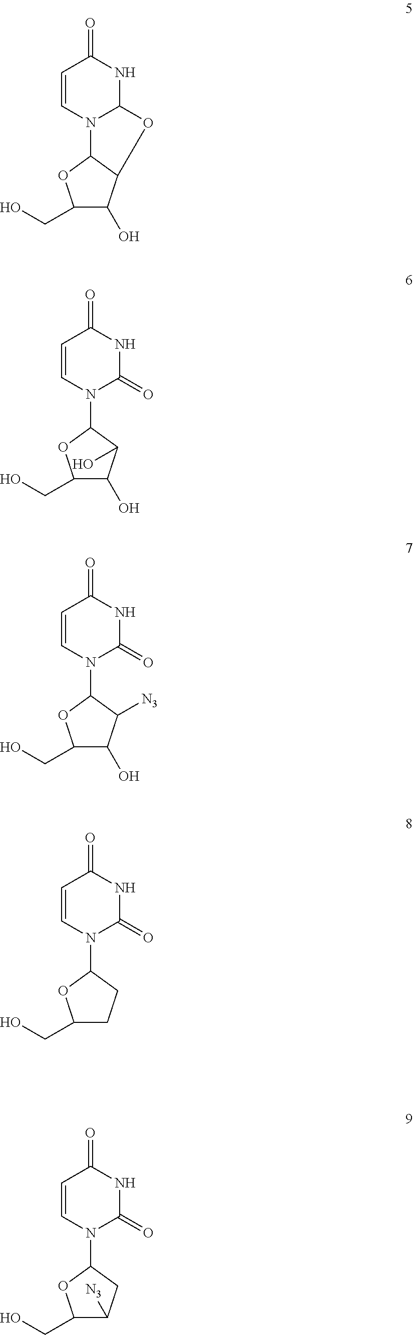

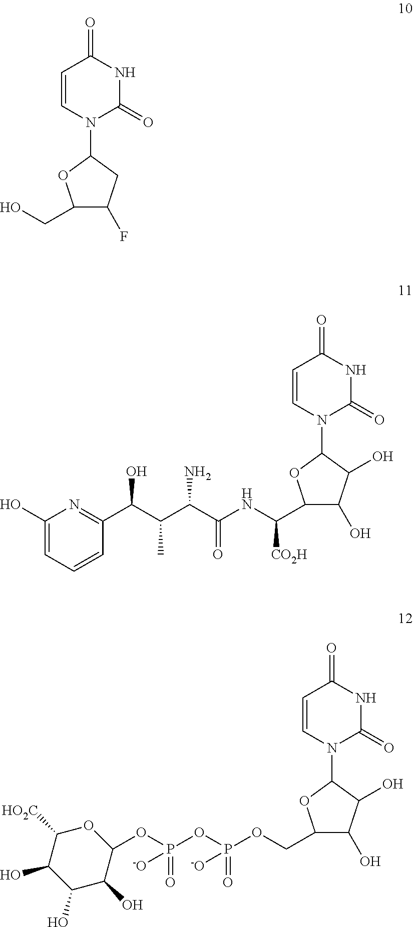

15. A compound according to any one of embodiments 1 to 14, wherein the disease ameliorated by the alteration of let-7 expression is selected from cancer or other proliferative conditions, coronary heart disease and diabetes.

### Administration

The compounds of formulae I, IIa or IIb or compounds identified in the assay described herein (the ‘active compounds’), or pharmaceutical compositions comprising these compounds, may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutang, gibbon), or a human.

Suitable formulations and dosage regimes for use with the compounds of formulae I, IIa or IIb or compounds identified in the assay described herein, are discussed in the Formulation and Dosage sections below.

### Formulations

While it is possible for an active compound described herein or identified by a method described herein, such as a modulator of miRNA activity, to be administered alone, it is preferable to present it as a pharmaceutical composition (e.g. formulation) comprising the active compound, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents. Such materials should be non-toxic and should not interfere with the efficacy of the active ingredient. The precise nature of the carrier or other material may depend on the route of administration as described below.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, Handbook of Pharmaceutical Additives⁵⁸, Remington's Pharmaceutical Sciences⁵⁹; and Handbook of Pharmaceutical Excipients⁶⁰.

The formulations may conveniently be presented in unit dosage form and may be prepared by any methods well-known in the art of pharmacy. Such methods include the step of bringing the active compound into association with a carrier which may constitute one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The active compound may be administered to a subject by any convenient route of administration, whether systemically/peripherally or at the site of desired action, including but not limited to, oral (e.g. by ingestion); topical (including e.g. direct administration to the CNS, for example, by intracranial injection or fusion); pulmonary (e.g. by inhalation or insufflation therapy using, e.g. an aerosol, e.g. through mouth or nose); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example, from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use.

Examples of techniques and protocols mentioned above can be found in Remington's Pharmaceutical Sciences⁵⁹.

### Dosage

It will be appreciated that appropriate dosages of the active compound can vary from patient to patient. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the active compound, other drugs, compounds, and/or materials used in combination, and the age, sex, weight, condition, general health, and prior medical history of the patient. The amount of active compound and route of administration will ultimately be at the discretion of the physician, although generally the dosage will be to achieve concentrations of the active compound at a site of therapy without causing substantial harmful or deleterious side-effects.

Administration in vivo can be effected in one dose, continuously or intermittently (e.g., in divided doses at appropriate intervals). Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the physician.

Active compounds may be administered in conjunction with other anti-cancer agents. Administration may be simultaneous, separate or sequential. By “simultaneous” administration, it is meant that a compound of the invention and a second anti-cancer agent are administered to a subject in a single dose by the same route of administration.

By “separate” administration, it is meant that a compound of the invention and a second anti-cancer agent are administered to a subject by two different routes of administration which occur at the same time. This may occur for example where one agent is administered by infusion and the other is given orally during the course of the infusion.

By “sequential” it is meant that the two agents are administered at different points in time, provided that the activity of the first administered agent is present and ongoing in the subject at the time the second agent is administered. For example, another anti-cancer agent may be administered first, such that tumour cells in the subject are damaged, followed by administration of the compound of the invention such that p53 function is provided to induce apoptosis. Generally, a sequential dose will occur such that the second of the two agents is administered within 48 hours, preferably within 24 hours, such as within 12, 6, 4, 2 or 1 hour(s) of the first agent.

The amount of the compound to be administered to a subject will ultimately depend upon the nature of the subject and the disease to be treated.

A second agent may be any known agent with desirable properties having regard to the disease to be treated. Such agents include taxoids such as Taxol®, Taxotere® or other chemotherapeutics, such as cis-platin (and other platin intercalating compounds), etoposide and etoposide phosphate, bleomycin, mitomycin C, CCNU, doxorubicin, daunorubicin, idarubicin, ifosfamide, and the like. The agent may also be a biological agent such as a protein that inhibits tumour growth, such as but not limited to interferon (IFN)-gamma, tumour necrosis factor (TNF)-alpha, TNF-beta, and similar cytokines, or an anti-angiogenic factor such as angiostatin and endostatin or inhibitors of FGF or VEGF such as soluble forms of receptors for angiogenic factors, including but not limited to soluble VGF/VEGF receptor.

### Methods

Another aspect of the invention provides a method of reducing cell proliferation or increasing cell differentiation comprising;
reducing the activity of ZCCHC poly(U)polymerase in the cell.

ZCCHC poly(U)polymerase activity may be reduced by treating the cell with an anti-ZCCHC antibody, RNAi molecule or anti-sense molecule, as described above.

ZCCHC poly(U)polymerase activity may be inhibited in vitro or in vivo, for example in the treatment of cancer.

Another aspect of the invention provides a method of inducing, stimulating or maintaining cell pluripotency comprising;
increasing the activity of ZCCHC poly(U)polymerase in the cell.

ZCCHC poly(U)polymerase may be stimulated in vitro or in vivo.

Various further aspects and embodiments of the present invention will be apparent to those skilled in the art in view of the present disclosure. All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described below.

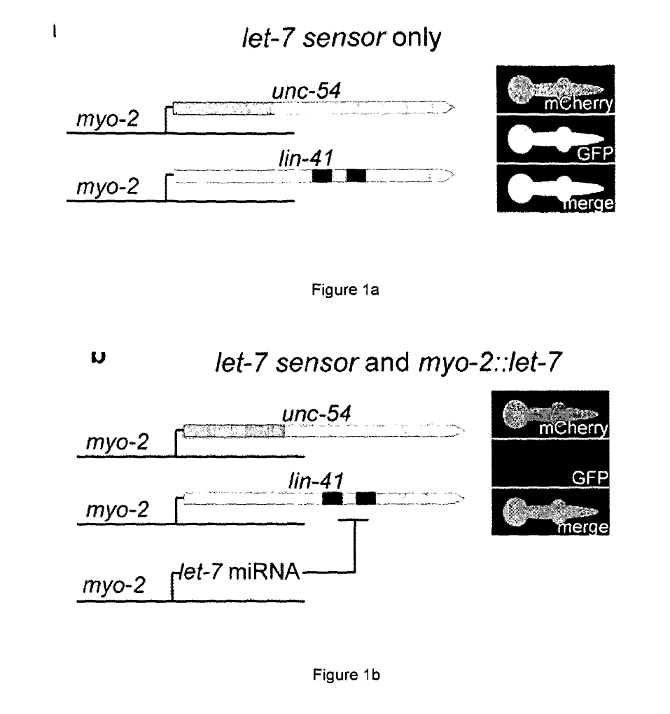

FIGS. 1a and 1b show a schematic of the pharynx based assay of let-7 activity.

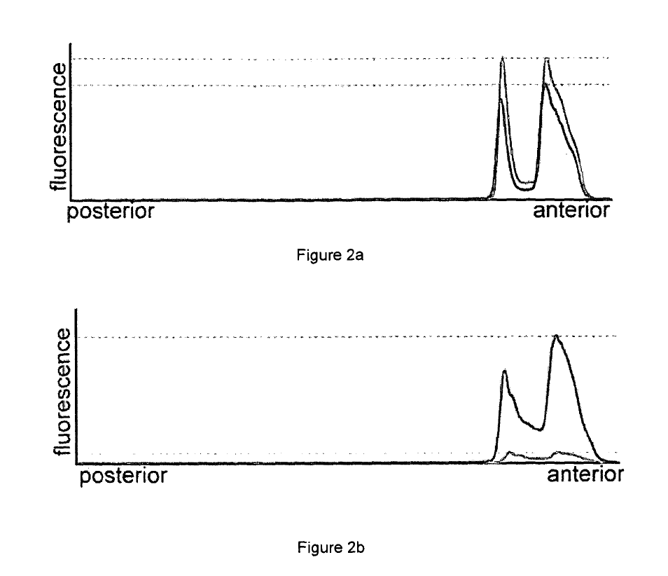

FIGS. 2a and 2b show a measurement of relative fluorescence by the COPAS Biosort instrument. Fluorescence profiles of representative individual L4 larval stage let-7 sensor only (2a), or let-7 sensor and myo-2::let-7 (2b) animals. Green and red lines indicate GFP and mCherry fluorescence respectively; dashed lines mark maximum intensity. In FIG. 2a the green line is the line having the highest relative fluorescence value (the top line). In FIG. 2b the red line in the line having the highest relative fluorescence value (the top line).

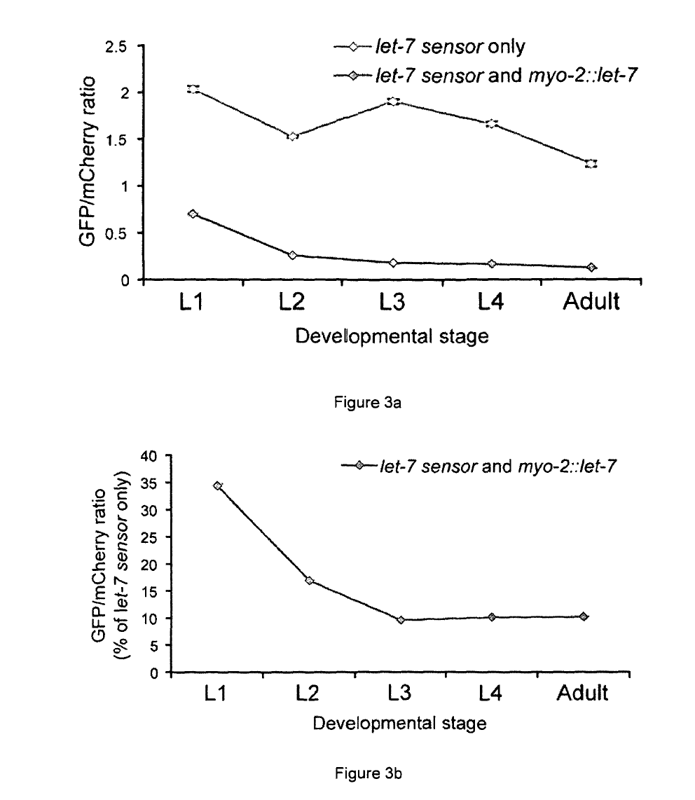

FIG. 3a shows relative GFP/mCherry ratio throughout larval development as measured by the COPAS Biosort instrument, n>400 for each larval stage, and n>70 for adults. Error bars show standard error of the mean. In FIG. 3a the top line corresponds to the GFP/mCherry ratio for the let-7 sensor only.

FIG. 3b shows quantification of myo-2::let-7 dependent regulation reveals developmental regulation of let-7 activity. GFP/mCherry ratio at each stage in let-7 sensor; myo-2::let-7 calculated as a percentage of that of let-7 sensor only animals. Error bars show standard error of the mean.

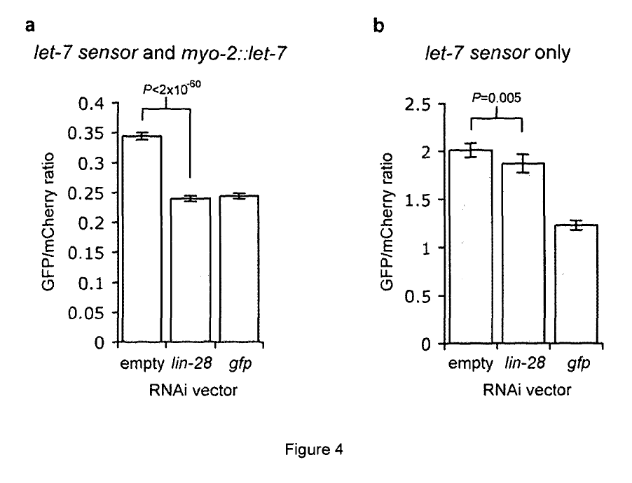

FIG. 4 shows the identification of lin-28 dependent regulation of let-7 activity by the COPAS Biosort instrument. lin-28 RNAi results in a decreased GFP/mCherry ratio in L2 larvae (a; P=2×10-61 Student's t-test, 2-tailed, equal variance, n>900). lin-28 RNAi results only in a slight reduction of the GFP/mCherry ratio in let-7 sensor only animals (b; P=0.005 Student's t-test, 2-tailed, equal variance, n>300). Error bars show standard error of the mean.

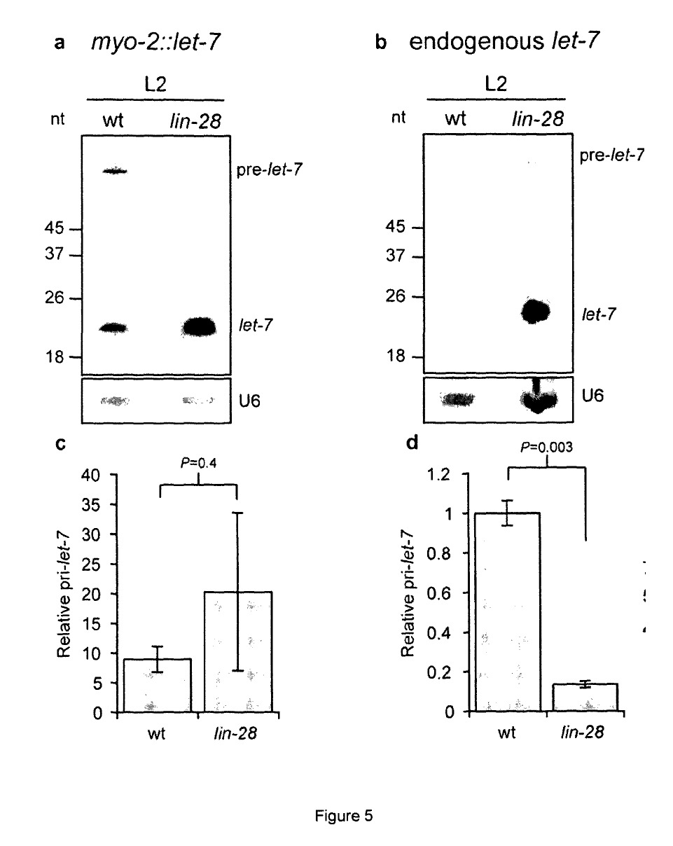

FIG. 5 shows that LIN-28 inhibits let-7 miRNA processing at the Dicer step and directly interacts with pre-let-7.

FIG. 5a shows Northern blot showing lin-28 dependent regulation of let-7 processing in myo-2::let-7 L2 larvae. 5 μg total RNA was loaded in each lane. U6 was used as a loading control. FIG. 5b shows Northern blot showing lin-28 dependent regulation of endogenous let-7 in L2 larvae. Small RNA fraction (miRVana) equivalent to 200 μg total RNA was loaded in each lane. U6 was used as a loading control. FIG. 5c shows RT-PCR for pri-let-7 in myo-2::let-7 animals. gpdh-2 mRNA was used as an internal control. pri-let-7 levels are unchanged in lin-28 mutants (P=0.4 Student's ttest, 2-tailed, equal variance, n=3 biological replicates). Error bars show standard error of the mean. FIG. 5d shows RT-PCR for endogenous pri-let-7. gpdh-2 mRNA was used as an internal control. pri-let-7 levels are reduced in lin-28 mutants (P=0.003, Student's t-test, 2-tailed, equal variance, n=3 biological replicates). Error bars show standard error of the mean.

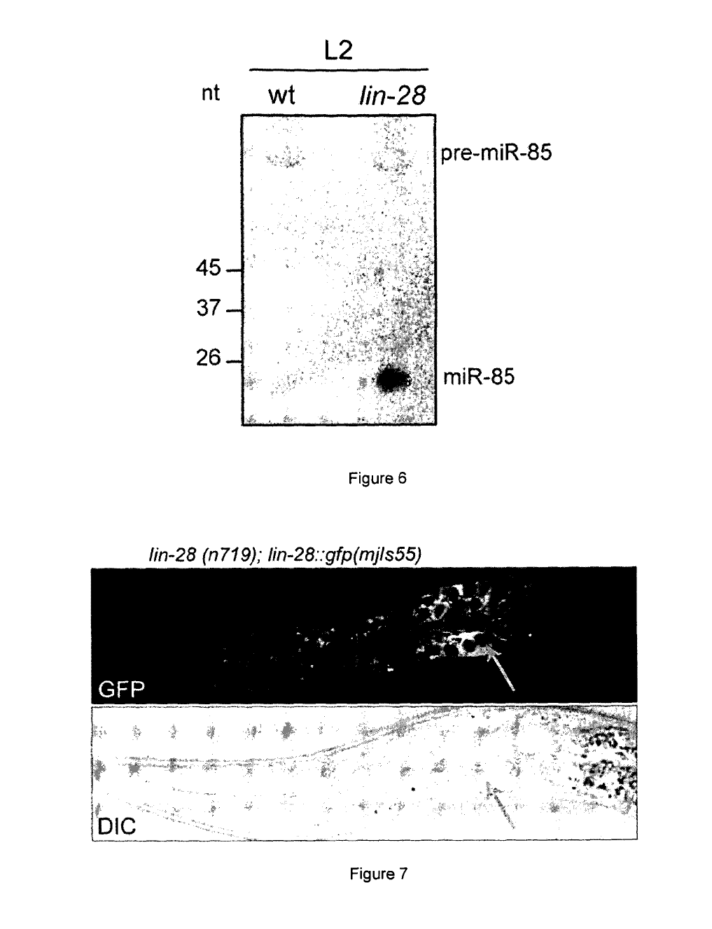

FIG. 6 shows Northern blot showing lin-28-dependent regulation of miR-85. The blot shown in 5b was stripped and re-probed.

FIG. 7 shows confocal image showing cytoplasmic localisation of LIN-28::GFP. Arrows indicate the nucleus of a representative cell in the pharynx.

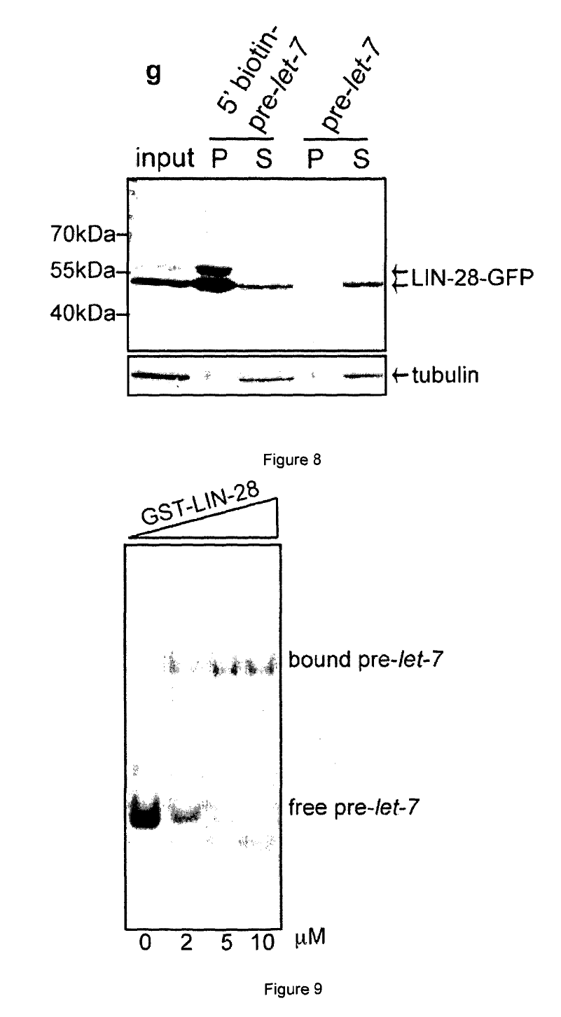

FIG. 8 shows pull-down of LIN-28::GFP using synthetic pre-let-7 showing that LIN-28 forms a complex with pre-let-7 (P; pull-down S; superantant). LIN-28 was detected as a doublet (arrows).

FIG. 9 shows electrophoretic mobility shift assay showing that LIN-28 binds directly to let-7 premiRNA in vitro.

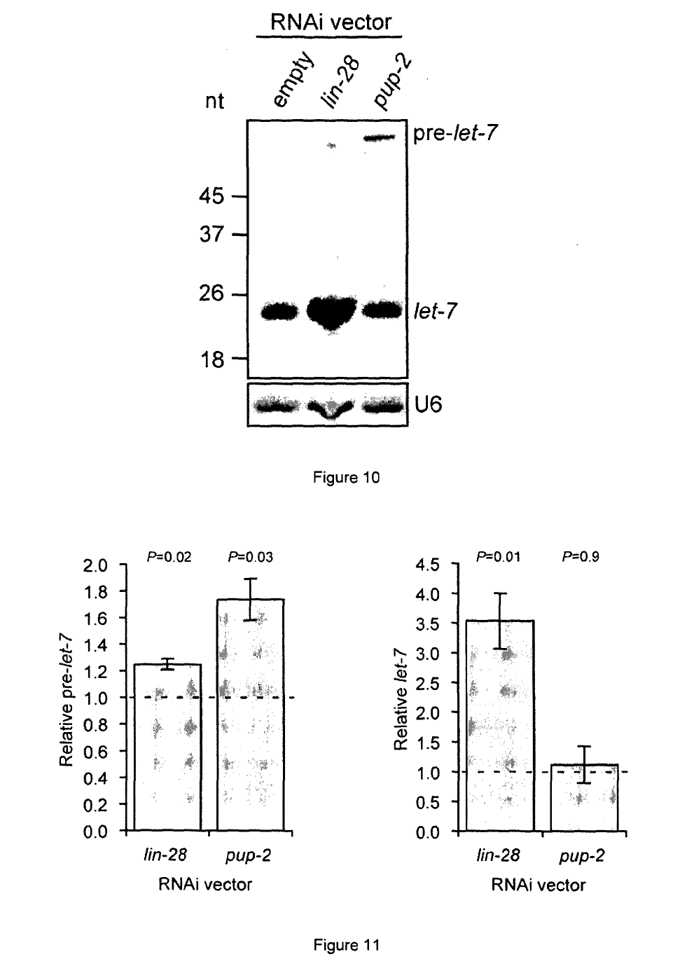

FIG. 10 shows a representative northern blot showing PUP2-dependent regulation of pre-let-7. 5g of total RNA from control, lin-28(RNAi), and PUP2(RNAi) myo-2::let-7 L2 larvae was loaded. U6 was used as a loading control.

FIG. 11 shows quantification of relative pre-let-7 (left panel), and let-7 (right panel) abundance in lin-28(RNAi) and PUP2(RNAi) myo-2::let-7 L2 larvae from independent northern blotting experiments. Mean fold change relative to empty vector control samples is shown. PUP2(RNAi) results in increased pre-let-7 whereas lin-28(RNAi) results in increased let-7 (P-values from single value t-tests indicated; n=3). Error bars show standard error of the mean.

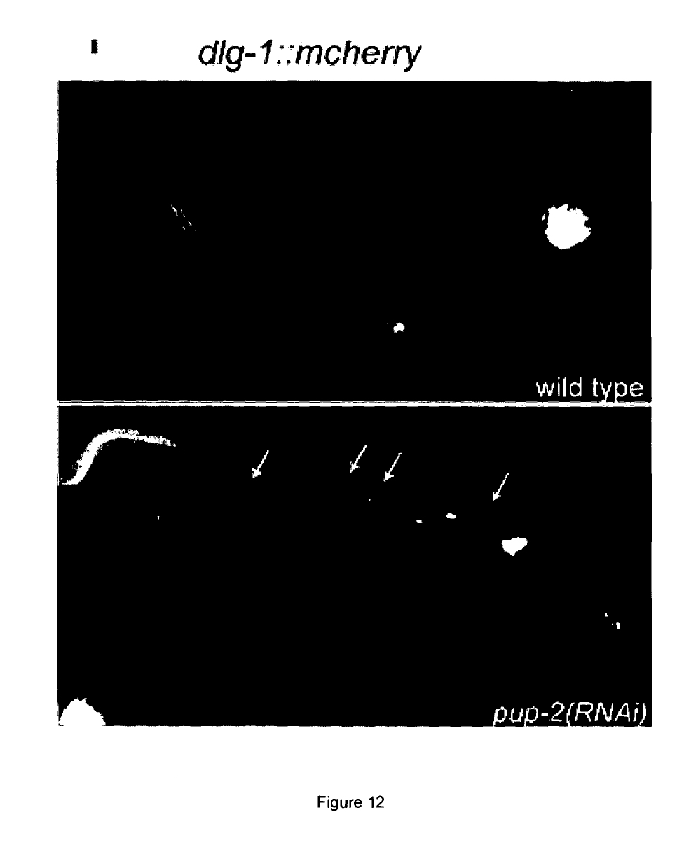

FIG. 12 shows a fluorescence image showing the seam cell defect observed in PUP2(RNAi) adults. A DLG-1-mCherry fusion marks seam cell boundaries. Upper panel; wild-type with continuous seam. Lower panel; PUP2(RNAi) with incompletely fused seam. Arrows indicate sites of failed fusion.

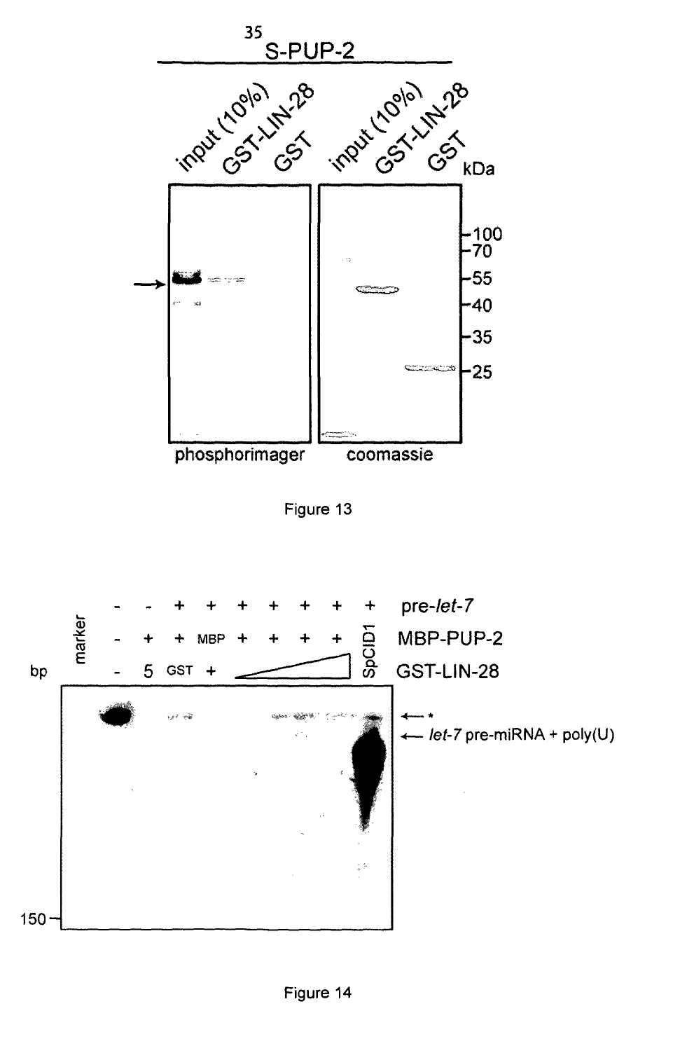

FIG. 13 shows GST pull-down assay demonstrating a direct interaction of GST-LIN-28 and PUP2 in vitro.

FIG. 14 shows a uridylation assay showing that PUP2 is a LIN-28 dependent pre-let-7 polyuridylase in vitro. * indicates unincorporated material aggregated in wells. Recombinant S. pombe CID1, which does not require tethering for activity in vitro, was used as a positive control.

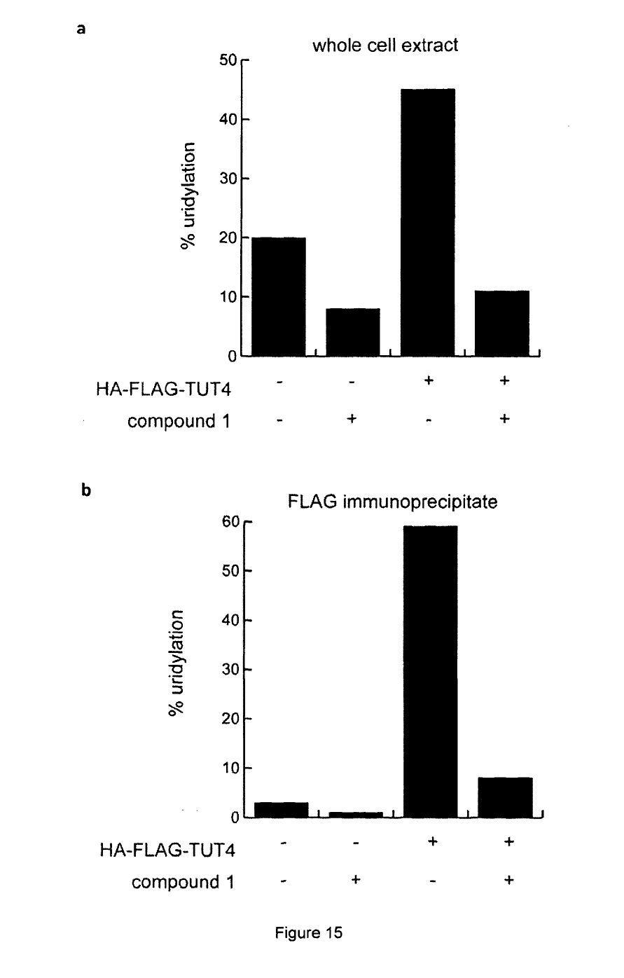

FIG. 15a shows the results of screening for inhibitors of the activity of the human ZCCHC polypeptide ZCCHC11 using whole cell extracts.

FIG. 15b shows the results of screening for inhibitors of activity of the human ZCCHC polypeptide ZCCHC11 using FLAG immunoprecipitates of ZCCHC11.

Table 1 shows the frequency of unmodified and modified let-7* molecules identified by high-throughput sequencing.

Table 2 shows the occurrence of seam cell defects in PUP2(RNAi) and lin-28(RNAi) adults.

### Experiments

### 1. Materials and Methods

### Nematode Culture and Strains

C, elegans was grown under standard conditions at 20° C. The food source used was E. coli strain HB101 (Caenorhabditis Genetics Center, University of Minnesota, Twin Cities, Minn., USA). Bleaching followed by starvation-induced L1 arrest was used to generate synchronized cultures. The wildtype strain was var. Bristol N2.

### DNA Constructs and Transgenics

All constructs were generated using the Multisite Gateway Three-Fragment vector construction kit (Invitrogen). Site directed mutagenesis was performed using PCR and mutagenic primers. All constructs were confirmed by sequencing. To generate transgenic animals, germline transformation was performed as described. Injection mixes contained 2-10 ng/μl of construct, 5-10 ng/μl of marker, and Invitrogen 1 kb ladder to a final concentration of 100 ng/μl DNA. Array transgenes were integrated via X-ray irradiation as described³⁴. Single copy transgenes were generated by transposase mediated integration (mosSCl) as described³⁵.

### Microscopy

Differential interference contrast (DIC) and fluorescence imaging was performed using standard methods³⁶ and using an Axiolmager A1 upright microscope (Zeiss, Jena, Germany). Images were captured using an ORCA-ER digital camera (Hamamatsu, Hamamatsu, Japan) and processed using OpenLabs 4.0 software (Improvision, Coventry, UK). For analysis of let-7 sensor transgene expression, images used for direct comparison were obtained and processed under identical conditions. Confocal microscopy was performed using an Olympus FluoView FV1000 upright microscope under 63× magnification.

Analyses with the COPAS Biosort Instrument

A COPAS Biosort instrument (Union Biometrica, Holliston, Mass., USA) was used to simultaneously measure length (time of flight), absorbance (extinction), and fluorescence. Fluorophore detection was optimised for simultaneous detection of GFP and mCherry. Excitation was achieved by multiline solid state argon laser (488 nm GFP and 561 nm mCherry), and emission detected by appropriate PMTs after passing through band pass filters (510/23 nm GFP and 615/45 nm mCherry). Animals were harvested from plates and washed in M9 buffer³¹ prior to sorting. Length and absorbance for each larval stage was determined using synchronised wild-type populations. These data were used to generate gates to isolate specific stages from mixed stage populations.

### RNA Interference Assays

RNAi clones were obtained from genome-wide RNAi libraries³⁷⁻³⁹. Additional RNAi constructs were generated by subcloning of an appropriate genomic DNA fragment into pDEST-L4440^{39,40}. All RNAi constructs were confirmed by sequencing. RNAi by feeding was performed as described using the eri-1(mg366) RNAi hypersensitive genetic background⁴¹. For COPAS Biosort analysis, 10-50 μl larvae were plated on 90 mm RNAi plates, and analysed once the oldest progeny reached the L3 larval stage. For harvest and RNA extraction, ˜3,000 L1 larvae were plated per RNAi plate, grown to adulthood, and bleached. After synchronisation by starvation, the progeny were placed onto fresh RNAi plates and grown to the desired stage before harvesting. RNAi by injection was performed as described⁴². Phenotypic analysis was performed on progeny laid 24-48 h post-injection.

### RNA Extraction

For total RNA isolation animals were harvested from plates by washing with M931. Animals were pelleted and frozen in liquid nitrogen and dissolved in ten pellet volumes of Trizol reagent (Invitrogen, Carlsbad, Calif., USA). Total RNA was extracted from Trizol reagent according to the manufacturer's protocol.

miRNA Microrarray Analysis

miRNA microarrays were performed using custom DNA oligonucleotide arrays as described previous^{43,44}. Data analysis was as described⁴⁴. To compare miRNA expression in wild-type and lin-28 mutant L2 larvae total RNA was isolated from synchronized animals and size selected to 18-26 nt using polyacrylamide gel electrophoresis. The small RNA fraction was 3′ end-labelled using T4 RNA ligase (Fermentas UK, York, UK). C. elegans miRNA microarrays were based on miRbase release 8.0^{45,46}. All experiments were performed in triplicates.

### Northern Blotting

Northern blotting was performed as described^{47,48}, with the following modifications. 5-20 μg total RNA, or small RNA fraction (miRvana, Ambion) isolated from ˜200 μg total RNA was used. For developmental expression profiles 1-ethyl-3-[3-dimethylaminopropyl]carbodimide hydrochloride (EDC, Perbio Science, Erembodegem, Belgium) crosslinking reactions were carried out for 2 h at 60° C. Otherwise blots were UV-crosslinked. Northern hybridisations were modified as follows; membranes were pre-hybridised at 40° C. for 4 h in hybridisation buffer (0.36 M Na2HPO4, 0.14 M NaH2PO4, 7% SDS and 1 mg of sheared, denatured salmon sperm DNA) and hybridised at 40° C. overnight using 20 pmole of γ-32P-ATP-radiolabelled DNA oligonucleotide probes. After hybridisation, membranes were washed twice with 0.5×SSC, 0.1% SDS at 40° C. for 10 min and once with 0.1×SSC, 0.1% SDS at 40° C. for 5 min. Radioactivity was detected by phosphoimager (GE Healthcare, Amersham, UK). Band intensity was quantified using ImageQuant software (GE Healthcare).

### Real-time RT-PCR

RT-PCR was performed as described⁴⁷, using the standard curve method.

pre-let-7 Pull-down

For these experiments we generated a strain carrying a rescuing lin-28::gfp translational fusion transgene (mosSCl integrated) in a lin-28(n719) mutant background. Protein extracts were prepared from starvation synchronised L1 larvae. Lysates were cleared against streptavadin Dynabeads (Invitrogen) for 30 min at 4° C. in PD buffer [18 mM HEPES-KOH pH 7.9, 10% glycerol, 40 mM KCl, 2 mM MgCl2, 10 mM DTT, 100 μM ZnSO4, 1× Proteinase Inhibitor Cocktail (PIC; Roche)]. Dynabeads were blocked with 15 μg yeast tRNA for 1 h at 4° C. in PD buffer before addition of 100 pmol synthetic 5′ biotinylated pre-let-7 (Microsynth, Balgach, Switzerland) for pull-down, or unmodified synthetic pre-let-7 for control reactions, and incubated for 1 h at room temperature. Pre-blocked Dynabeads were added to the binding reaction and incubated for 1 h at room temperature. Beads were washed three times in PD buffer. Bound proteins were analysed by western blotting with primary mouse anti-GFP (Clontech JL-8; 1:1000) and secondary HRP-conjugated anti-mouse (Dakocytomation P0450; 1:10,000), and rat anti-tubulin (Chemicon international MAB1684, 1:1000) and secondary HRP-conjugated mouse anti-rat (GE Healthcare NA9310; 1:10,000).

### Recombinant Protein Expression

LIN-28 cDNA (F02E9.2b) was obtained from the ORFeome library³⁹. The PUP2 cDNA clone was a kind gift of M. Wickens³⁰. cDNAs were subcloned into pDEST-GEX-2TK (Gateway cassette inserted at SmaI site in pGEX-2TK), or pDEST-MAL (a gift from J. Pines). Recombinant proteins were expressed and purified as described^{47,48}.

### GST-Pull-Down

PUP2 cDNA was subcloned into pDEST14 (Invitrogen), and ³⁵Smethionine-radiolabelled protein was produced by in vitro transcription-translation using a TNT T7 coupled reticulocyte lysate kit (Promega). Pull-downs were performed using GST-LIN28 as described⁴⁹.

### Pre-let-7 Transcription

In vitro transcription reactions were performed in a volume of 20 μl with 0.5 mM of each NTP, 40 mM Tris pH 7.9, 12 mM MgCl2, 2 mM spermidine, 20 mM DTT, 1 mM NaCl, 100 U T7 RNA polymerase (Roche, Basel, Switzerland), and 1U RNasin (Promega, Madison Wis., USA). Reactions were incubated for 1 h at 37° C., phenol/chloroform extracted and ethanol precipitated.

Radiolabeled RNA for electrophoretic mobility shift assays was transcribed with α-³²P-UTP to a specific activity of approximately 6,000 cpm/fmol.

### In Vitro Uridylation Assays

In vitro uridylation assays were performed in 30 μl reactions containing 1.5 μg of in vitro transcribed pre-let-7 in 10 mM Tris pH 7.5, 30 mM KCl, 1 mM DTT, 10 mM MnCl2, 2 mM MgCl2, 0.25 mM UTP, 1 μl of RNaseOut and 0.01 Mbq α-32P-UTP. 1 μg of recombinant MBP-PUP2 and increasing amounts of recombinant GST-LIN-28 were added to a maximum of 10 μg.

The reaction mixtures were incubated at 30° C. for 30 min. RNA was purified by phenol/chloroform extraction and ethanol precipitated. Reactions were analysed in a 6% urea polyacrylamide gel. 2U S. pombe CID1 poly(U) polymerase (NEB, Ipswich, Mass., USA) was used as a positive control. Radioactivity was detected by phosphoimager (GE Healthcare).

### Electrophoretic Mobility Shift Assay

Binding reactions were carried out in a total volume of 20 μl containing 50,000 cpm of radiolabelled RNA, 30 μg tRNA, 1 μl RNaseOut (40 unit/μl, Invitrogen), 50 mM Tris pH 7.6, 100 mM NaCl, 0.07% β-mercaptoethanol, 5 mM MgOAc2, and increasing amounts of recombinant GST-LIN-28 to a maximum of 10 μM. The reactions were incubated at room temperature for 45 min, followed by analysis using 5% native polyacrylamide gel electrophoresis. Radioactivity was detected by phosphoimager (GE Healthcare).

### Screening Assay for PUP2 Inhibitors

HEK293T cells were transfected with pHA-FLAG-ZCCHC11. After 48 hrs cells were lysed. 50 μl of the supernatant was incubated with 5 μl of pre-washed anti-FLAG antibody conjugated to agarose beads (Sigma) and incubated for 2 hrs at 4° C. Agarose-beads were washed twice with lysis buffer and twice with buffer D. For in vitro uridylation, agarose beads were incubated in a 30 μl reaction containing 3.2 mM of MgCl₂, 1 mM of DTT and 0.25 mM of rUTP, 5 mM of test compound and 5′-end-labeled pre-let-7 of 1×10⁴-1×10⁵ cpm, for 20 min at 37° C. RNA was purified by Trizol extraction and isopropanol precipitated. Reactions were analysed in a 12% urea polyacrylamide gel.

### 2. Results

We established a quantitative miRNA reporter assay based on let-7 in C. elegans (FIG. 1a,b). We generated two transgenes comprising the promoter of myo-2, the coding sequences of either GFP or mCherry and the 3′UTR of either lin-41 or unc-54 (myo-2::gfp::lin-41 and myo-2::mcherry::unc-54; hereafter referred to as the let-7 sensor, FIG. 1a). The myo-2 promoter confers expression exclusively in the pharyngeal muscle of C. elegans²⁴, and lin-41 is a genetically identified target of the let-7 miRNA19, whereas the unc-54 3′UTR is not known to be regulated by any miRNA. Transgenic animals carrying an intrachromosomal array of the let-7 sensor expressed both GFP and mCherry strongly throughout larval development and adult stages.

let-7 is not known to be expressed in the pharynx and these data showed that endogenous let-7 miRNA did not regulate the let-7 sensor. In contrast, in animals carrying an additional transgene expressing let-7 from the myo-2 promoter GFP expression was inhibited, while mCherry expression was unaffected (myo-2::let-7). Surprisingly, this effect was developmentally regulated with a markedly stronger inhibition of GFP expression in adults than L1 larvae. As the let-7 transgene did not contain the let-7 promoter, the let-7 activity must be regulated after transcription.

We used a COPAS Biosort instrument to quantify GFP and mCherry expression along the body axis of thousands of individual animals. Using the Biosort instrument the peaks of GFP and mCherry expression in the two pharyngeal bulbs of let-7 sensor transgenic animals are apparent (FIG. 2a, 2b). Besides fluorescence emission, the Biosort instrument records length and absorbance of individual animals, which we used to calculate developmental stage. Animals carrying the myo-2::let-7 transgene in addition to the let-7 sensor showed a reduced ratio of GFP/mCherry relative to animals carrying the let-7 sensor only; this effect was most marked at later stages (FIG. 3a). To compare the extent of myo-2::let-7 mediated silencing between stages, we calculated relative GFP/mCherry ratios for the two transgenic strains for each developmental stage (FIG. 3b). let-7 sensor silencing was least efficient at the L1 larval stage, during which the GFP/mCherry ratio was reduced by approximately 65%. let-7 sensor mediated silencing increased during development, reaching maximal efficiency during the L3 larval stage, when the GFP/mCherry ratio was reduced by 90%. This developmental pattern correlates strikingly with the temporal expression pattern of endogenous let-7 miRNA, which begins to accumulate during the L3 stage3. We therefore postulated that these data reflect a mechanism that post-transcriptionally regulates the activity or accumulation of let-7 to ensure appropriately timed downregulation of let-7 targets during development.

Next we carried out forward genetic screens and an RNAi screen to identify factors regulating let-7 activity in vivo. We found that knockdown of lin-28 by RNAi resulted in reduced GFP/mCherry ratios at the L1 and L2 stages compared to an empty vector control (FIG. 4a). This effect was dependent on let-7 as lin-28 RNAi had little effect on animals carrying the let-7 sensor only (FIG. 4b). In contrast, knockdown of gfp by RNAi reduced GFP/mCherry ratios independent of the let-7 transgene (FIG. 4a,b). We independently confirmed these results using a loss-of-function mutation in lin-28. In lin-28 (n719) mutants the let-7 sensor is silenced at all developmental stages tested and this silencing is dependent on the expression of let-7. Mutations in lin-46 completely suppress the developmental timing defect of lin-28 mutants²⁵, however developmental regulation of let-7 activity was not restored in lin-28; lin-46 double mutants. Thus the deregulation of let-7 activity in lin-28 mutants is not an indirect result of developmental timing defects. In addition, a number of other heterochronic genes, including lin-14 and lin-42 did not affect the let-7 sensor. LIN-28 expression is restricted to the L1 and L2 stages during larval development¹⁸.

Next we tested if LIN-28 was sufficient to inhibit let-7 activity. Ectopic expression of LIN-28 in the pharynx from an extrachromosomal array resulted in the inhibition of let-7 function in adult animals. Mosaic expression of the extrachromosomal array within the pharynx indicated that LIN-28 acted cell autonomously. We concluded that LIN-28 is required and sufficient to inhibit let-7 activity in C. elegans.

We analysed miRNA expression in wild-type and lin-28 mutant L2 larvae using miRNA microarrays. We found let-7 to be significantly (approximately 15-fold) overexpressed in lin-28 mutant L2 larvae, while other members of the let-7 family were not significantly overexpressed. Three unrelated miRNAs, including miR-85, were also significantly overexpressed in lin-28 mutant L2 larvae. Like let-7, miR-85 is also developmentally regulated²⁷. We confirmed the microarray results for let-7, let-7 family members and miR-85 by northern blotting.

We sought to address whether Lin28 regulates let-7 processing at the Drosha^{7,9} or Dicer^{6,10} level in vivo in C. elegans. First, we used northern blotting to compare let-7 expression from the myo-2::let-7 transgene in otherwise wild-type and lin-28 mutant L2 larvae. lin-28 mutants expressed higher levels of let-7 compared to wild type, indicating increased processing efficiency; this was accompanied by a slight reduction in the level of prelet-7, consistent with increased efficiency of Dicer-mediated processing (FIG. 5a).

We obtained similar results for endogenous let-7, although pre-let-7 levels were not reduced in this case (FIG. 5b). Next, we determined pri-let-7 levels by RT-PCR. In animals carrying the myo-2::let-7 transgene abundance of pri-let-7 was slightly increased in lin-28 mutants compared to wild type, but this was not significant (P=0.4; FIG. 5c). Endogenous levels of pri-let-7 were decreased in lin-28 mutants (FIG. 5d). These data did not support LIN-28 regulation at the Drosha step. We obtained similar results for miR-85 (FIG. 6). Consistent with these findings we found that a functional LIN-28-GFP translational fusion is expressed in the cytoplasm and excluded from the nucleus in all cell types inspected, including in the pharynx (FIG. 7). Taken together, these data provide indication that LIN-28 blocks Dicer mediated processing of the developmentally regulated miRNAs let-7 and mir-85.

Next, we tested whether LIN-28 acts by directly interacting with pre-let-7. First, we tested whether pre-let-7 associates with LIN-28 from whole animal lysates. We mixed lysates from animals carrying a transgene expressing LIN-28 fused to GFP with synthetic pre-let-7. We then performed pull-down assays using streptavidin beads. LIN-28-GFP was retained on streptavidin beads if the synthetic pre-let-7 RNA was biotinylated, but not using a non-biotinylated control (FIG. 8). Second, we tested whether this interaction was direct using a native gel mobility shift assay using recombinant GST-LIN-28 and in vitro transcribed pre-/et-7. We found that pre-let-7 interacts with GST-LIN-28 with an estimated Kd of 2 μM (FIG. 9). We conclude that LIN-28 acts by directly regulating pre-let-7 processing.

We tested a number of pre-let-7 loop mutants in vivo using the let-7 sensor. We found that the pre-let-7 loop is not required for the normal developmental regulation of let-7 activity.

Our results so far were consistent with a LIN-28 blockade of pre-let-7 processing. However, pre-let-7 did not accumulate significantly at the L2 larval stage in wild-type as compared to lin-28 mutant animals (FIG. 5a,b). We inspected published high-throughput sequencing data of C. elegans small RNA libraries²⁹, and found frequent modification of the 3′ end of let-7* with 1 or 2 untemplated uracil residues (C. elegans let-7 resides on the 5′ arm of the hairpin; Table 1). These may result from Dicer processing of partially uridylated intermediates. We carried out an RNAi screen against potential poly(U) polymerases assaying let-7 and pre-let-7 abundance in myo-2::let-7 transgenic L2 larvae. RNAi against a single gene, PUP2, resulted in increased pre-let-7 levels (FIG. 10). However, mature let-7 levels were not altered (FIG. 11).

These data provide indication that PUP2 uridylation targets pre-let-7 for degradation, but this is not rate limiting for blocking let-7 processing in this transgenic background.

Consistent with this finding, PUP2 RNAi did not significantly affect let-7 sensor expression. We therefore sought a sensitive assay to determine if PUP2 is required for the correct regulation of let-7 activity in vivo. Precocious expression of let-7 results in altered timing of larval development and defects in the differentiation of a hypodermal stem cell lineage required for the formation of lateralalae, a cuticle structure in adult animals¹⁴. Lateral seam cells differentiate and fuse into a syncytium in wild-type adults, but this fusion is defective if PUP2 or lin-28 are knocked down, consistent with a role in regulating let-7 (Table 2, FIG. 12). Knockdown of PUP2 and lin-28 together did not enhance this effect, providing indication that both act in the same pathway. These data show that PUP2 contributes to robust execution of the developmental timing pathway.

Next we sought direct evidence that PUP2 mediates LIN-28-dependent uridylation of pre-let-7. Using GST pull-down experiments we found that PUP2 directly interacts with GST-LIN-28 in vitro (FIG. 13). PUP2 was previously shown to polyuridylate an artificially tethered RNA in Xenopus oocytes³⁰. Therefore, we tested if LIN-28 might be able to recruit PUP2 to mediate pre-let-7 uridylation (FIG. 14). A tethering-independent poly (U) polymerase, SpCID1, was sufficient to polyuridylate pre-let-7 in vitro. In contrast, PUP2 was inactive on its own and required the addition of LIN-28 to polyuridylate pre-let-7. Here we demonstrate that LIN-28 recruits the poly (U) polymerase PUP2 to uridylate C. elegans pre-let-7.

Next, we screened a panel of test compounds for inhibitors of ZCCHC polyuridylation activity. The screening method is described in more detail above.

The effect of a test compound (“compound 1”) on endogenous and HA-FLAG-ZCCHC11 uridylyl activities in whole cell extracts are shown in FIG. 15a and in FLAG immunoprecipitates in FIG. 15b. It can be seen that the test compound reduces the uridylation activity of both whole cell extracts and FLAG immunoprecipitates of ZCCHC11.

The above results show that ZCCHC poly(U) polymerase regulates the stability of let-7 pre-miRNA under LIN-28 control. ZCCHC poly(U) polymerase and LIN-28 are shown to interact directly, and LIN-28 is shown to stimulate uridylation of let-7 pre-miRNA by the ZCCHC poly(U) polymerase in vitro. In addition, ZCCHC poly (U) polymerase is shown to contribute to regulation of a stem cell lineage in vivo. These results demonstrate that LIN-28 and let-7 form an ancient regulatory switch, which is conserved from nematodes to human, and provide insight into the mechanism of LIN-28 action in vivo. Uridylation by a ZCCHC poly(U) polymerase might regulate let-7 and additional miRNAs in other species. Given the roles of Lin28 and let-7 in stem cell and cancer biology, ZCCHC poly(U) polymerases may therefore represent therapeutic targets.

TABLE 1

| | | n clones |
|---|---|---|
| | 5′ CUAUGCAAUUUUCACCUUACC 3′ | 23 |
| let-7* | 5′ CUAUGCAAUUUUCACCUUACCU 3′ | 11 |
| | 5′ CUAUGCAAUUUUCACCUUACCUU 3′ | 2 |
| let-7 genomic | 5′ ACCGGTGAACTATGCAATTTTCACCT | |
| | TACCGG 3′ | |

TABLE 2

| treatment | incomplete seam (%) | n |
|---|---|---|
| uninjected control | 0 | 156 |
| lin-28(RNAi) | 24.49 | 49 |
| pup-2(RNAi) | 2.30 | 217 |
| pup-2(RNAi); lin-28(RNAi) | 22.50 | 40 |

### 3. Assay

Screening Assay for ZCHCC poly(U) Polymerase Inhibitors

HEK293T cells were transfected with pHA-FLAG-ZCCHC11. After 48 hrs cells were lysed. 50 μl of the supernatant was incubated with 5 μl of pre-washed anti-FLAG antibody conjugated to agarose beads (Sigma) and incubated for 2 hrs at 4° C. Agarose-beads were washed twice with lysis buffer and twice with buffer D. For in vitro uridylation, agarose beads were incubated in a 30 μl reaction containing 3.2 mM of MgCl₂, 1 mM of DTT and 0.25 mM of rUTP, 5 mM of test compound and 5′-end-labeled pre-let-7 of 1×10⁴-1×10⁵ cpm, for 20 min at 37° C. RNA was purified by Trizol extraction and isopropanol precipitated. Reactions were analysed in a 12% urea polyacrylamide gel.

### 4. Results

The effect of a test compound (“compound 1”) on endogenous and HA-FLAG-ZCCHC11 uridylyl activities in whole cell extracts are shown in FIG. 15a and in FLAG immunoprecipitates in FIG. 15b. It can be seen that the test compound reduces the uridylation activity of both whole cell extracts and FLAG immunoprecipitates of ZCCHC11.

Compound 1 (uridine 5′-diphosphoglucuronic acid (UDP-GlcA)) is shown below:

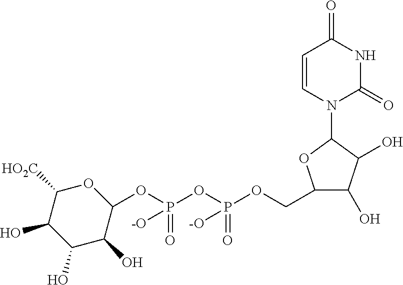

The compound was used as the trisodium salt.

### REFERENCES

The following references are incorporated by reference herein in their entirety:
1 Johnson, C. D. et al. Cancer Res 67, 7713-7722 (2007).
2 Johnson, S. M. et al. Cell 120, 635-647 (2005).
3 Reinhart, B. J. et al. Nature 403, 901-906 (2000).
4 Yu, F. et al. Cell 131, 1109-1123 (2007).
5 Pasquinelli, A. E. et al. Nature 408, 86-89 (2000).
6 Heo, I. et al. Mol Cell 32, 276-284 (2008).
7 Viswanathan, S. R. et al. Science 320, 97-100 (2008).
8 Yu, J. et al. Science 318, 1917-1920 (2007).
9 Newman, M. A. et al RNA 14, 1539-1549 (2008).
10 Rybak, A. et al. Nat Cell Biol 10, 987-993 (2008).
11 Bartel, D. P. Cell 116, 281-297 (2004).
12 Bartel, D. P. Cell 36, 215-233 (2009).
13 Kim, V. N. et al Nat ev Mol Cell Biol 10, 126-139 (2009).
14 Ambros, V. & Horvitz, H. R. Science 226, 409-416 (1984).
Chalfie, M. et al Cell 24, 59-69 (1981).
16 Lee, R. C. et al. Cell 75 843-854 (1993).
17 Wightman, B. et al Cell 75, 855-862 (1993).
18 Moss, E. G. et al Cell 88, 637-646 (1997).
19 Slack, F. J. et al. Mol Cell 5, 659-669 (2000).
20 Abrahante, J. E. et al. Dev Cell 4, 625-637 (2003).
21 Grosshans, H. et al Dev Cell 8, 321-330 (2005).
22 Lin, S. Y. et al. Dev Cell 4, 639-650 (2003).
23 Winter, J. et al Nat Cell Biol 11, 228-234 (2009).
24 Miller, D. M. et al Proc Natl Acad Sci USA 83, 2305-2309 (1986).
25 Pepper, A. S. et al. Development 131, 2049-2059 (2004).
26 Bracht, J. et al. RNA 10, 1586-1594 (2004).
27 Lim, L. P. et al. Genes Dev 17 991-1008 (2003).
28 Piskounova, E. et al. J Biol Chem 283 21310-21314 (2008).
29 Batista, P. J. et al Mol Cell 31, 67-78 (2008).
30 Kwak, J. E. et al RNA 13, 860-867 (2007).
31 Wood, W. The Nematode Caenorhabditis elegans. (Cold Spring Harbour Press, 1988).
32 Brenner, S. Genetics 77, 71-94 (1974).
33 Mello, C. et al Methods Cell Biol 48, 451-482 (1995).
34 Fire, A. EMBO J. 5, 2673-2680 (1986).
Frøkjaer-Jensen, C, et al. Nat Genet. 40, 1375-1383 (2008).
36 Horvitz, H. R. et al. Genetics 96, 435-454 (1980).
37 Fraser, A. G. et al. Nature 408, 325-330 (2000).
38 Kamath, R. S. et al. Nature 421, 231-237 (2003).
39 Rual, J. F. et al. Genome Res 14, 2162-2168 (2004).
40 Timmons, L. et al 263, 103-112 (2001).
41 Kennedy, S. et al. Nature 427, 645-649 (2004).
42 Fire, A. et al. Nature 391, 806-811 (1998).
43 Miska, E. A. at al. Genome Biol 5, R68 (2004).
44 Wienholds, E. et al. Science 309, 310-311 (2005).
45 Griffiths-Jones, S, Nucleic Acids Res 32, D109-111 (2004).
46 Griffiths-Jones, S. et al. Nucleic Acids Res 34, D140-144 (2006).
47 Das, P. P. et al. Mol Cell 31, 79-90 (2008).
48 Pall, G. S. et at Nat Protoc 3, 1077-1084 (2008).
49 Sapetschnig, A. et al EMBO J. 21, 5206-5215 (2002).
50 Cho Molecular Cancer (2007) 6:60
51 Zhang et al Developmental Biology 302 (2007) 1-12
52 Viswanathan et al Nature Genetics online publn. 31 May 2009
53 Garofalo et al Current Opinion in Pharmacology 2008, 8:661-667
54 Büssing, I., et al., Trends in Molecular Medicine, 14(9), 400-410 (2008).
55 Schickel, R., at al., Oncogene, 27, 5959-5974 (2008).
56 Berge, et al., “Pharmaceutically Acceptable Salts”, J. Pharm. Sci., 66, 1-19 (1977).
57 Protective Groups in Organic Synthesis, T. Green and P. Wuts; 3rd Edition; John Wiley and Sons, 1999).
58 Handbook of Pharmaceutical Additives, 2nd Edition (eds. M. Ash and I. Ash), 2001 (Synapse Information Resources, Inc., Endicott, N.Y., USA).
59 Remington's Pharmaceutical Sciences, 20th edition, pub. Lippincott, Williams & Wilkins, 2000.
60 Handbook of Pharmaceutical Excipients, 2nd edition, 1994.
61 Evan et al., Mol. Cell. Biol. 5, 3610-3616 (1985).
62 Fields & Song Nature 340, 245-246 (1989).
63 Altschul et al. (1990) J. Mol. Biol. 215: 405-410.
64 Pearson and Lipman (1988) PNAS USA 85: 2444-2448.
65 Smith and Waterman (1981) J. Mol. Biol. 147: 195-197.
66 Altschul et al. Nucl. Acids Res. (1997) 25 3389-3402.
67 Peyman and Ulman, Chemical Reviews, 90:543-584, (1990).
68 Crooke, Ann. Rev. Pharmacol. Toxicol. 32:329-376, (1992).
69 Zamore P D et al Cell, 101, 25-33, (2000).
70 Elbashir S M. et al. Nature, 411, 494-498, (2001).
71 Kashani-Sabet and Scanlon, 1995, Cancer Gene Therapy, 2(3): 213-223.
72 Mercola and Cohen, 1995, Cancer Gene Therapy, 2(1), 47-59.
73 Angell & Baulcombe (1997) The EMBO Journal 16, 12:3675-3684.
74 Voinnet & Baulcombe (1997) Nature 389: pg 553.
75 Fire A. et al Nature 391, (1998).
76. Zamore P. D. Nature Structural Biology, 8, 9, 746-750, (2001).

1. A method of identifying a compound which modulates miRNA activity comprising:
determining the ability of a test compound to alter the polyuridylation activity of a ZCCHC polypeptide,
wherein a test compound which alters the polyuridylation activity is useful in modulating miRNA activity.

2. A method according to claim 1 wherein a test compound which alters the polyuridylation activity is useful in modulating the activity of a human miRNA.

3. A method according to claim 1 wherein the ability of the test compound to alter the polyuridylation activity is determined by contacting the ZCCHC polypeptide with an RNA molecule in the presence and absence of the test compound and measuring the uridylation of the RNA molecule by the ZCCHC polypeptide.

4. A method according to claim 3 wherein the RNA molecule is a pre-miRNA.

5. A method according to claim 4 wherein the RNA molecule is a pre-let-7 miRNA.

6. A method according to claim 1 wherein a difference in the polyuridylation activity of a ZCCHC polypeptide in the presence relative to the absence of the test compound is indicative that the test compound modulates miRNA activity.

7. A method according to claim 1 wherein a test compound which alters the polyuridylation activity of a ZCCHC polypeptide may be useful in modulating miRNA activity.

8. A method according to claim 3 wherein the polyuridylation of the RNA molecule by the ZCCHC polypeptide is determined in the presence of a LIN28 polypeptide.

9. A method of identifying a compound which modulates miRNA activity comprising:
determining the ability of a test compound to alter the binding of a ZCCHC polypeptide to a LIN28 polypeptide,
wherein a test compound which alters said binding may be useful in modulating miRNA activity.

10. A method according to claim 9 wherein the ZCCHC polypeptide and the LIN28 polypeptide are contacted in the presence and absence of a test compound.

11. A method according to claim 10 wherein a difference in the binding of the ZCCHC polypeptide and the LIN28 polypeptide in the presence relative to the absence of a test compound is indicative that the compound modulates miRNA activity.

12. A method according to claim 9 wherein a test compound which alters said binding may be useful in modulating human miRNA activity

13. A method according to claim 9 wherein the LIN28 polypeptide comprises an amino acid sequence having at least 30% sequence identity to SEQ ID NO: 20 or SEQ ID NO: 22.

14. A method of identifying a compound which modulates miRNA activity comprising:
determining the ability of a test compound to bind to a ZCCHC polypeptide,
wherein a test compound which binds to the ZCCHC polypeptide may be useful in modulating miRNA activity.

15. A method according to claim 14 wherein a test compound which alters said binding may be useful in modulating human miRNA activity.

16. A method according to claim 1 wherein the ZCCHC polypeptide comprises an amino acid sequence having at least 30% sequence identity to SEQ ID NO: 2.

17. A method according to claim 1 wherein the ZCCHC polypeptide is a ZCCHC11 polypeptide which comprises an amino acid sequence having at least 30% sequence identity to any one of SEQ ID NOS: 3 to 8.

18. A method according to claim 1 wherein the ZCCHC polypeptide is a ZCCHC6 polypeptide which comprises an amino acid sequence having at least 30% sequence identity to any one of SEQ ID NOS: 9 to 18.

19. A method according to claim 1 comprising identifying a test compound which modulates miRNA activity.

20. A method according to claim 19 further comprising determining the effect of said identified compound on biological activity of an miRNA.

21-24. (canceled)


SEQUENCE LISTING (26 sequences)

SEQ ID NO: 1 - DNA, 2365 bp, Caenorhabditis elegans
agtatggagtgctagaaaagctcaaacacgcggtacaacacacacattcactggacaccactgaccacaagatccctcacatcaactgacacttactaatgacagttattcagaagtcgtctcctacagtgaaaggagtgaagacaacaacatcagattgcaccgaaagacgtcactcatcgagttcaccttgcaaaatgagtcaaaacggaagcagtggatactatgaaggagatgttttactggatgatgatgctcttgtttcgatgccagaagcaacgctcattgagtgctttggtaatctggctgtcacaaaaagacctgcacatctgagacctccggcagctttctgtctaccgtttgtatcaatgaacaacgatttgacgagcggacatgccttgaaacatatcacattgacatcagtattcgacgcactacatccgtacgttgccatgccgtacatcaacagaatggatatatgctctgcaatgaacgtatattggatcagaaactgtttggaagctgaacaatccgatttatttcatcgattcgctctggaaatgcaagtacatttgagtgcatgttttggatgtcgcgttgtcctggatatctatggatcaaccagaaacggattcggtacccgattctgtgatgttgatatgtcactttctttttcaccgagtccaccttcatgggcaaccaattcggatcgagtgatgagagctgttgcgaaggcacttgtcgattttccgaaagcagttgatgagagatatgtcaacgccaaagttccaattgtcagattcagaagcagtgacatggatatggaagcagacatcagttacaagaatgatttggctcttcacaatactcagcttttgcaacaatactgcaaatgggatcccgaaagactaccgactcttggagtttgggtcaaagcgtgggcgaaacgaagtggagttggagatgcatcaaaaggatcgctgtcttcatatgcttggattgtgatgcttattcattatcttcagcaagtcgaaccaattcctgtgttgccatgtcttcaggaaatgaaccatcagaaaagtgaaaatgtctacgtccaaggttacaacacgtattattggaaattcgtagacactgctcgtacacgacgttgtcgtgcttctgtcgttgatttgttcgttggattcttggattactatgccacatactttgactactccacaaatgttatccagatggtgtccaagaaattggaattcaaaccggatcgctggtgcaagtatccaatgtgtattgctgatccgttcgagacggatcataatttggcacaaggtgttgatatgccaatgtttgaatacattagatcgtgtatggaacactcgaagaaagtattcacggatcgccgcatgcgctcggagtttctttcgggatacgggttcgacgttgacgaatttgatgcaaggcatcgaggcgaaatgaacatggaaatggcgtcacaatttggggaattccttcttcacaagtgcataatggtgaaacaagccccgaatcgtcaattccgtgatcgcagtatgagtcaatcgacgagcataagcaacacatcgagtatttcctcgtcgggatagtttttcatacgtgtttcttctcatcatcatcatttttttatcgaagaacggtatccaggagctttttttataatttgtcaaaactcccgggtattcattctttctcttttctaacctaacctttacaaaccgatcacaaaatttattaattacccctttattaaccttttttatcacaagtcctagtgattcctagtgcatccattgcccattttaattgtatatgtacataattgaaccccatccccacccgacatcacaacctcatcaatttttctcaaaacttttcaaaaatcgcgccttttcttcccatttttgtgggtcttgatattcacacacacagccggctctgtattcaaaaaatctcaaaaaatcatgcgcaaagtcacttttctctccatgtgtcccctcatttgaacaaaaaaaatgtataaaatcacaagagctggttctcggaaattgttttcacccgtttatgtattttcatctcccttctcatatttccctgtgtgaatgtaaattgactcactaaattaatttctttattccacctgatttcgagactaataagtctgagcttaattaccctccctgcatccaaattccccaaataatattaacgatcaagttttatcaactgttgatttttctttctacggctctcttaaaatattcaagtccctcctcactaattccctccccccccccctttttttcttttattacatacgaatttttct

SEQ ID NO: 2 - protein, 508 aa, Caenorhabditis elegans
MTVIQKSSPTVKGVKTTTSDCTERRHSSSSPCKMSQNGSSGYYEGDVLLDDDALVSMPEATLIECFGNLAVTKRPAHLRPPAAFCLPFVSMNNDLTSGHALKHITLTSVFDALHPYVAMPYINRMDICSAMNVYWIRNCLEAEQSDLFHRFALEMQVHLSACFGCRVVLDIYGSTRNGFGTRFCDVDMSLSFSPSPPSWATNSDRVMRAVAKALVDFPKAVDERYVNAKVPIVRFRSSDMDMEADISYKNDLALHNTQLLQQYCKWDPERLPTLGVWVKAWAKRSGVGDASKGSLSSYAWIVMLIHYLQQVEPIPVLPCLQEMNHQKSENVYVQGYNTYYWKFVDTARTRRCRASVVDLFVGFLDYYATYFDYSTNVIQMVSKKLEFKPDRWCKYPMCIADPFETDHNLAQGVDMPMFEYIRSCMEHSKKVFTDRRMRSEFLSGYGFDVDEFDARHRGEMNMEMASQFGEFLLHKCIMVKQAPNRQFRDRSMSQSTSISNTSSISSSG

SEQ ID NO: 3 - protein, 1644 aa, Homo sapiens
MEESKTLKSENHEPKKNVICEESKAVQVIGNQTLKARNDKSVKEIENSSPNRNSSKKNKQNDICIEKTEVKSCKVNAANLPGPKDLGLVLRDQSHCKAKKFPNSPVKAEKATISQAKSEKATSLQAKAEKSPKSPNSVKAEKASSYQMKSEKVPSSPAEAEKGPSLLLKDMRQKTELQQIGKKIPSSFTSVDKVNIEAVGGEKCALQNSPRSQKQQTCTDNTGDSDDSASGIEDVSDDLSKMKNDESNKENSSEMDYLENATVIDESALTPEQRLGLKQAEERLERDHIFRLEKRSPEYTNCRYLCKLCLIHIENIQGAHKHIKEKRHKKNILEKQEESELRSLPPPSPAHLAALSVAVIELAKEHGITDDDLRVRQEIVEEMSKVITTFLPECSLRLYGSSLTRFALKSSDVNIDIKFPPKMNHPDLLIKVLGILKKNVLYVDVESDFHAKVPVVVCRDRKSGLLCRVSAGNDMACLTTDLLTALGKIEPVFIPLVLAFRYWAKLCYIDSQTDGGIPSYCFALMVMFFLQQRKPPLLPCLLGSWIEGFDPKRMDDFQLKGIVEEKFVKWECNSSSATEKNSIAEENKAKADQPKDDTKKTETDNQSNAMKEKHGKSPLALETPNRVSLGQLWLELLKFYTLDFALEEYVICVRIQDILTRENKNWPKRRIAIEDPFSVKRNVARSLNSQLVYEYVVERFRAAYRYFACPQTKGGNKSTVDFKKREKGKISNKKPVKSNNMATNGCILLGETTEKINAEREQPVQCDEMDCTSQRCIIDNNNLLVNELDFADHGQDSSSLSTSKSSEIEPKLDKKQDDLAPSETCLKKELSQCNCIDLSKSPDPDKSTGTDCRSNLETESSHQSVCTDTSATSCNCKATEDASDLNDDDNLPTQELYYVFDKFILTSGKPPTIVCSICKKDGHSKNDCPEDFRKIDLKPLPPMTNRFREILDLVCKRCFDELSPPCSEQHNREQILIGLEKFIQKEYDEKARLCLFGSSKNGFGFRDSDLDICMTLEGHENAEKLNCKEIIENLAKILKRHPGLRNILPITTAKVPIVKFEHRRSGLEGDISLYNTLAQHNTRMLATYAAIDPRVQYLGYTMKVFAKRCDIGDASRGSLSSYAYILMVLYFLQQRKPPVIPVLQEIFDGKQIPQRMVDGWNAFFFDKTEELKKRLPSLGKNTESLGELWLGLLRFYTEEFDFKEYVISIRQKKLLTTFEKQWTSKCIAIEDPFDLNHNLGAGVSRKMTNFIMKAFINGRKLFGTPFYPLIGREAEYFFDSRVLTDGELAPNDRCCRVCGKIGHYMKDCPKRKSLLFRLKKKDSEEEKEGNEEEKDSRDVLDPRDLHDTRDFRDPRDLRCFICGDAGHVRRECPEVKLARQRNSSVAAAQLVRNLVNAQQVAGSAQQQGDQSIRTRQSSECSESPSYSPQPQPFPQNSSQSAAITQPSSQPGSQPKLGPPQQGAQPPHQVQMPLYNFPQSPPAQYSPMHNMGLLPMHPLQIPAPSWPIHGPVIHSAPGSAPSNIGLNDPSIIFAQPAARPVAIPNTSHDGHWPRTVAPNSLVNSGAVGNSEPGFRGLTPPIPWEHAPRPHFPLVPASWPYGLHQNFMHQGNARFQPNKPFYTQDRCATRRCRERCPHPPRGNVSE

SEQ ID NO: 4 - protein, 907 aa, Homo sapiens
MKSEKVPSSPAEAEKGPSLLLKDMRQKTELQQIGKKIPSSFTSVDKVNIEAVGGEKCALQNSPRSQKQQTCTDNTGDSDDSASGIEDVSDDLSKMKNDESNKENSSEMDYLENATVIDESALTPEQRLGLKQAEERLERDHIFRLEKRSPEYTNCRYLCKLCLIHIENIQGAHKHIKEKRHKKNILEKQEESELRSLPPPSPAHLAALSVAVIELAKEHGITDDDLRVRQEIVEEMSKVITTFLPECSLRLYGSSLTRFALKSSDVNIDIKFPPKMNHPDLLIKVLGILKKNVLYVDVESDFHAKVPVVVCRDRKSGLLCRVSAGNDMACLTTDLLTALGKIEPVFIPLVLAFRYWAKLCYIDSQTDGGIPSYCFALMVMFFLQQRKPPLLPCLLGSWIEGFDPKRMDDFQLKGIVEEKFVKWECNSSSATEKNSIAEENKAKADQPKDDTKKTETDNQSNAMKEKHGKSPLALETPNRVSLGQLWLELLKFYTLDFALEEYVICVRIQDILTRENKNWPKRRIAIEDPFSVKRNVARSLNSQLVYEYVVERFRAAYRYFACPQTKGGNKSTVDFKKREKGKISNKKPVKSNNMATNGCILLGETTEKINAEREQPVQCDEMDCTSQRCIIDNNNLLVNELDFADHGQDSSSLSTSKSSEIEPKLDKKQDDLAPSETCLKKELSQCNCIDLSKSPDPDKSTGTDCRSNLETESSHQSVCTDTSATSCNCKATEDASDLNDDDNLPTQELYYVFDKFILTSGKPPTIVCSICKKDGHSKNDCPEDFRKIDLKPLPPMTNRFREILDLVCKRCFDELSPPCSEQHNREQILIGLEKFIQKEYDEKARLCLFGSSKNGFGFRDSDLDICMTLEGHENAEALIVGRRLSKLQGRKRLIGQGRSVPGLLNSN

SEQ ID NO: 5 - protein, 309 aa, Homo sapiens
MEESKTLKSENHEPKKNVICEESKAVQVIGNQTLKARNDKSVKEIENSSPNRNSSKKNKQNDICIEKTEVKSCKVNAANLPGPKDLGLVLRDQSHCKAKKFPNSPVKAEKATISQAKSEKATSLQAKAEKSPKSPNSVKAEKASSYQMKSEKVPSSPAEAEKGPSLLLKDMRQKTELQQIGKKIPSSFTSVDKVNIEAVGGEKCALQNSPRSQKQQTCTDNTGDSDDSASGIEDVSDDLSKMKNDESNKENSSEMDYLENATVIDESALTPEQRLGLKQAEERLERDHIFRLEKVYYVVLVIWGEMCVS

SEQ ID NO: 6 - protein, 1645 aa, Homo sapiens
MEESKTLKSENHEPKKNVICEESKAVQVIGNQTLKARNDKSVKEIENSSPNRNSSKKNKQNDICIEKTEVKSCKVNAANLPGPKDLGLVLRDQSHCKAKKFPNSPVKAEKATISQAKSEKATSLQAKAEKSPKSPNSVKAEKASSYQMKSEKVPSSPAEAEKGPSLLLKDMRQKTELQQIGKKIPSSFTSVDKVNIEAVGGEKCALQNSPRSQKQQTCTDNTGDSDDSASGIEDVSDDLSKMKNDESNKENSSEMDYLENATVIDESALTPEQRLGLKQAEERLERDHIFRLEKRSPEYTNCRYLCKLCLIHIENIQGAHKHIKEKRHKKNILEKQEESELRSLPPPSPAHLAALSVAVIELAKEHGITDDDLRVRQEIVEEMSKVITTFLPECSLRLYGSSLTRFALKSSDVNIDIKFPPKMNHPDLLIKVLGILKKNVLYVDVESDFHAKVPVVVCRDRKSGLLCRVSAGNDMACLTTDLLTALGKIEPVFIPLVLAFRYWAKLCYIDSQTDGGIPSYCFALMVMFFLQQRKPPLLPCLLGSWIEGFDPKRMDDFQLKGIVEEKFVKWECNSSSATEKNSIAEENKAKADQPKDDTKKTETDNQSNAMKEKHGKSPLALETPNRVSLGQLWLELLKFYTLDFALEEYVICVRIQDILTRENKNWPKRRIAIEDPFSVKRNVARSLNSQLVYEYVVERFRAAYRYFACPQTKGGNKSTVDFKKREKGKISNKKPVKSNNMATNGCILLGETTEKINAEREQPVQCDEMDCTSQRCIIDNNNLLVNELDFADHGQDSSSLSTSKSSEIEPKLDKKQDDLAPSETCLKKELSQCNCIDLSKSPDPDKSTGTDCRSNLETESSHQSVCTDTSATSCNCKATEDASDLNDDDNLPTQELYYVFDKFILTSGKPPTIVCSICKKDGHSKNDCPEDFRKIDLKPLPPMTNRFREILDLVCKRCFDELSPPCSEQHNREQILIGLEKFIQKEYDEKARLCLFGSSKNGFGFRDSDLDICMTLEGHENAEKLNCKEIIENLAKILKRHPGLRNILPITTAKVPIVKFEHRRSGLEGDISLYNTLAQHNTRMLATYAAIDPRVQYLGYTMKVFAKRCDIGDASRGSLSSYAYILMVLYFLQQRKPPVIPVLQEIFDGKQIPQRMVDGWNAFFFDKTEELKKRLPSLGKNTESLGELWLGLLRFYTEEFDFKEYVISIRQKKLLTTFEKQWTSKCIAIEDPFDLNHNLGAGVSRKMTNFIMKAFINGRKLFGTPFYPLIGREAEYFFDSRVLTDGELAPNDRCCRVCGKIGHYMKDCPKRKSSLLFRLKKKDSEEEKEGNEEEKDSRDVLDPRDLHDTRDFRDPRDLRCFICGDAGHVRRECPEVKLARQRNSSVAAAQLVRNLVNAQQVAGSAQQQGDQSIRTRQSSECSESPSYSPQPQPFPQNSSQSAAITQPSSQPGSQPKLGPPQQGAQPPHQVQMPLYNFPQSPPAQYSPMHNMGLLPMHPLQIPAPSWPIHGPVIHSAPGSAPSNIGLNDPSIIFAQPAARPVAIPNTSHDGHWPRTVAPNSLVNSGAVGNSEPGFRGLTPPIPWEHAPRPHFPLVPASWPYGLHQNFMHQGNARFQPNKPFYTQDRCATRRCRERCPHPPRGNVSE

SEQ ID NO: 7 - protein, 907 aa, Homo sapiens; identical to SEQ ID NO: 4

SEQ ID NO: 8 - protein, 1640 aa, Homo sapiens
MEESKTLKSENHEPKKNVICEESKAVQVIGNQTLKARNDKSVKEIENSSPNRNSSKKNKQNDICIEKTEVKSCKVNAANLPGPKDLGLVLRDQSHCKAKKFPNSPVKAEKATISQAKSEKATSLQAKAEKSPKSPNSVKAEKASSYQMKSEKVPSSPAEAEKGPSLLLKDMRQKTELQQIGKKIPSSFTSVDKVNIEAVGGEKCALQNSPRSQKQQTCTDNTGDSDDSASGIEDVSDDLSKMKNDESNKENSSEMDYLENATVIDESALTPEQRLGLKQAEERLERDHIFRLEKRSPEYTNCRYLCKLCLIHIENIQGAHKHIKEKRHKKNILEKQEESELRSLPPPSPAHLAALSVAVIELAKEHGITDDDLRVRQEIVEEMSKVITTFLPECSLRLYGSSLTRFALKSSDVNIDIKFPPKMNHPDLLIKVLGILKKNVLYVDVESDFHAKVPVVVCRDRKSGLLCRVSAGNDMACLTTDLLTALGKIEPVFIPLVLAFRYWAKLCYIDSQTDGGIPSYCFALMVMFFLQQRKPPLLPCLLGSWIEGFDPKRMDDFQLKGIVEEKFVKWECNSSSATEKNSIAEENKAKADQPKDDTKKTETDNQSNAMKEKHGKSPLALETPNRVSLGQLWLELLKFYTLDFALEEYVICVRIQDILTRENKNWPKRRIAIEDPFSVKRNVARSLNSQLVYEYVVERFRAAYRYFACPQTKGGNKSTVDFKKREKGKISNKKPVKSNNMATNGCILLGETTEKINAEREQPVQCDEMDCTSQRCIIDNNNLLVNELDFADHGQDSSSLSTSKSSEIEPKLDKKQDDLAPSETCLKKELSQCNCIDLSKSPDPDKSTGTDCRSNLETESSHQSVCTDTSATSCNCKATEDASDLNDDDNLPTQELYYVFDKFILTSGKPPTIVCSICKKDGHSKNDCPEDFRKIDLKPLPPMTNRFREILDLVCKRCFDELSPPCSEQHNREQILIGLEKFIQKEYDEKARLCLFGSSKNGFGFRDSDLDICMTLEGHENAEKLNCKEIIENLAKILKRHPGLRNILPITTAKVPIVKFEHRRSGLEGDISLYNTLAQHNTRMLATYAAIDPRVQYLGYTMKVFAKRCDIGDASRGSLSSYAYILMVLYFLQQRKPPVIPVLQEIFDGKQIPQRMVDGWNAFFFDKTEELKKRLPSLGKNTESLGELWLGLLRFYTEEFDFKEYVISIRQKKLLTTFEKQWTSKCIAIEDPFDLNHNLGAGVSRKMTNFIMKAFINGRKLFGTPFYPLIGREAEYFFDSRVLTDGELAPNDRCCRVCGKIGHYMKDCPKRKRLKKKDSEEEKEGNEEEKDSRDVLDPRDLHDTRDFRDPRDLRCFICGDAGHVRRECPEVKLARQRNSSVAAAQLVRNLVNAQQVAGSAQQQGDQSIRTRQSSECSESPSYSPQPQPFPQNSSQSAAITQPSSQPGSQPKLGPPQQGAQPPHQVQMPLYNFPQSPPAQYSPMHNMGLLPMHPLQIPAPSWPIHGPVIHSAPGSAPSNIGLNDPSIIFAQPAARPVAIPNTSHDGHWPRTVAPNSLVNSGAVGNSEPGFRGLTPPIPWEHAPRPHFPLVPASWPYGLHQNFMHQGNARFQPNKPFYTQDRCATRRCRERCPHPPRGNVSE

SEQ ID NO: 9 - protein, 1495 aa, Homo sapiens
MGDTAKPYFVKRTKDRGTMDDDDFRRGHPQQDYLIIDDHAKGHGSKMEKGLQKKKITPGNYGNTPRKGPCAVSSNPYAFKNPIYSQPAWMNDSHKDQSKRWLSDEHTGNSDNWREFKPGPRIPVINRQRKDSFQENEDGYRWQDTRGCRTVRRLFHKDLTSLETTSEMEAGSPENKKQRSRPRKPRKTRNEENEQDGDLEGPVIDESVLSTKELLGLQQAEERLKRDCIDRLKRRPRNYPTAKYTCRLCDVLIESIAFAHKHIKEKRHKKNIKEKQEEELLTTLPPPTPSQINAVGIAIDKVVQEFGLHNENLEQRLEIKRIMENVFQHKLPDCSLRLYGSSCSRLGFKNSDVNIDIQFPAIMSQPDVLLLVQECLKNSDSFIDVDADFHARVPVVVCREKQSGLLCKVSAGNENACLTTKHLTALGKLEPKLVPLVIAFRYWAKLCSIDRPEEGGLPPYVFALMAIFFLQQRKEPLLPVYLGSWIEGFSLSKLGNFNLQDIEKDVVIWEHTDSAAGDTGITKEEAPRETPIKRGQVSLILDVKHQPSVPVGQLWVELLRFYALEFNLADLVISIRVKELVSRELKDWPKKRIAIEDPYSVKRNVARTLNSQPVFEYILHCLRTTYKYFALPHKITKSSLLKPLNAITCISEHSKEVINHHPDVQTKDDKLKNSVLAQGPGATSSAANTCKVQPLTLKETAESFGSPPKEEMGNEHISVHPENSDCIQADVNSDDYKGDKVYHPETGRKNEKEKVGRKGKHLLTVDQKRGEHVVCGSTRNNESESTLDLEGFQNPTAKECEGLATLDNKADLDGESTEGTEELEDSLNHFTHSVQGQTSEMIPSDEEEEDDEEEEEEEEPRLTINQREDEDGMANEDELDNTYTGSGDEDALSEEDDELGEAAKYEDVKECGKHVERALLVELNKISLKEENVCEEKNSPVDQSDFFYEFSKLIFTKGKSPTVVCSLCKREGHLKKDCPEDFKRIQLEPLPPLTPKFLNILDQVCIQCYKDFSPTIIEDQAREHIRQNLESFIRQDFPGTKLSLFGSSKNGFGFKQSDLDVCMTINGLETAEGLDCVRTIEELARVLRKHSGLRNILPITTAKVPIVKFFHLRSGLEVDISLYNTLALHNTRLLSAYSAIDPRVKYLCYTMKVFTKMCDIGDASRGSLSSYAYTLMVLYFLQQRNPPVIPVLQEIYKGEKKPEIFVDGWNIYFFDQIDELPTYWSECGKNTESVGQLWLGLLRFYTEEFDFKEHVISIRRKSLLTTFKKQWTSKYIVIEDPFDLNHNLGAGLSRKMTNFIMKAFINGRRVFGIPVKGFPKDYPSKMEYFFDPDVLTEGELAPNDRCCRICGKIGHFMKDCPMRRKVRRRRDQEDALNQRYPENKEKRSKEDKEIHNKYTEREVSTKEDKPIQCTPQKAKPMRAAADLGREKILRPPVEKWKRQDDKDLREKRCFICGREGHIKKECPQFKGSSGSLSSKYMTQGKASAKRTQQES

SEQ ID NO: 10 - protein, 395 aa, Homo sapiens
MTINGLETAEGLDCVRTIEELARVLRKHSGLRNILPITTAKVPIVKFFHLRSGLEVDISLYNTLALHNTRLLSAYSAIDPRVKYLCYTMKVFTKIYKGEKKPEIFVDGWNIYFFDQIDELPTYWSECGKNTESVGQLWLGLLRFYTEEFDFKEHVISIRRKSLLTTFKKQWTSKYIVIEDPFDLNHNLGAGLSRKMTNFIMKAFINGRRVFGIPVKGFPKDYPSKMEYFFDPDVLTEGELAPNDRCCRICGKIGHFMKDCPMRRKVRRRRDQEDALNQRYPENKEKRSKEDKEIHNKYTEREVSTKEDKPIQCTPQKAKPMRAAADLGREKILRPPVEKWKRQDDKDLREKRCFICGREGHIKKECPQFKGSSGSLSSKYMTQGKASAKRTQQES

SEQ ID NO: 11 - protein, 784 aa, Homo sapiens
MGNEHISVHPENSDCIQADVNSDDYKGDKVYHPETGRKNEKEKVGRKGKHLLTVDQKRGEHVVCGSTRNNESESTLDLEGFQNPTAKECEGLATLDNKADLDGESTEGTEELEDSLNHFTHSVQGQTSEMIPSDEEEEDDEEEEEEEEPRLTINQREDEDGMANEDELDNTYTGSGDEDALSEEDDELGEAAKYEDVKECGKHVERALLVELNKISLKEENVCEEKNSPVDQSDFFYEFSKLIFTKGKSPTVVCSLCKREGHLKKDCPEDFKRIQLEPLPPLTPKFLNILDQVCIQCYKDFSPTIIEDQAREHIRQNLESFIRQDFPGTKLSLFGSSKNGFGFKQSDLDVCMTINGLETAEGLDCVRTIEELARVLRKHSGLRNILPITTAKVPIVKFFHLRSGLEVDISLYNTLALHNTRLLSAYSAIDPRVKYLCYTMKVFTKMCDIGDASRGSLSSYAYTLMVLYFLQQRNPPVIPVLQEIYKGEKKPEIFVDGWNIYFFDQIDELPTYWSECGKNTESVGQLWLGLLRFYTEEFDFKEHVISIRRKSLLTTFKKQWTSKYIVIEDPFDLNHNLGAGLSRKMTNFIMKAFINGRRVFGIPVKGFPKDYPSKMEYFFDPDVLTEGELAPNDRCCRICGKIGHFMKDCPMRRKVRRRRDQEDALNQRYPENKEKRSKEDKEIHNKYTEREVSTKEDKPIQCTPQKAKPMRAAADLGREKILRPPVEKWKRQDDKDLREKRCFICGREGHIKKECPQFKGSSGSLSSKYMTQGKASAKRTQQES

SEQ ID NO: 12 - protein, 1259 aa, Homo sapiens
MGDTAKPYFVKRTKDRGTMDDDDFRRGHPQQDYLIIDDHAKGHGSKMEKGLQKKKITPGNYGNTPRKGPCAVSSNPYAFKNPIYSQPAWMNDSHKDQSKRWLSDEHTGNSDNWREFKPGPRIPVINRQRKDSFQENEDGYRWQDTRGCRTVRRLFHKDLTSLETTSEMEAGSPENKKQRSRPRKPRKTRNEENEQDGDLEGPVIDESVLSTKELLGLQQAEERLKRDCIDRLKRRPRNYPTAKYTCRLCDVLIESIAFAHKHIKEKRHKKNIKEKQEEELLTTLPPPTPSQINAVGIAIDKVVQEFGLHNENLEQRLEIKRIMENVFQHKLPDCSLRLYGSSCSRLGFKNSDVNIDIQFPAIIEGFSLSKLGNFNLQDIEKDVVIWEHTDSAAGDTGITKEEAPRETPIKRGQVSLILDVKHQPSVPVGQLWVELLRFYALEFNLADLVISIRVKELVSRELKDWPKKRIAIEDPYSVKRNVARTLNSQPVFEYILHCLRTTYKYFALPHKITKSSLLKPLNAITCISEHSKEVINHHPDVQTKDDKLKNSVLAQGPGATSSAANTCKVQPLTLKETAESFGSPPKEEMGNEHISVHPENSDCIQADVNSDDYKGDKVYHPETGRKNEKEKVGRKGKHLLTVDQKRGEHVVCGSTRNNESESTLDLEGFQNPTAKECEGLATLDNKADLDGESTEGTEELEDSLNHFTHSVQGQTSEMIPSDEEEEDDEEEEEEEEPRLTINQREDEDGMANEDELDNTYTGSGDEDALSEEDDELGEAAKYEDVKECGKHVERALLVELNKISLKEENVCEEKNSPVDQSDFFYEFSKLIFTKGKGLDCVRTIEELARVLRKHSGLRNILPITTAKVPIVKFFHLRSGLEVDISLYNTLALHNTRLLSAYSAIDPRVKYLCYTMKVFTKMCDIGDASRGSLSSYAYTLMVLYFLQQRNPPVIPVLQEIYKGEKKPEIFVDGWNIYFFDQIDELPTYWSECGKNTESVGQLWLGLLRFYTEEFDFKEHVISIRRKSLLTTFKKQWTSKYIVIEDPFDLNHNLGAGLSRKMTNFIMKAFINGRRVFGIPVKGFPKDYPSKMEYFFDPDVLTEGELAPNDRCCRICGKIGHFMKDCPMRRKVRRRRDQEDALNQRYPENKEKRSKEDKEIHNKYTEREVSTKEDKPIQCTPQKAKPMRAAADLGREKILRPPVEKWKRQDDKDLREKRCFICGREGHIKKECPQFKGSSGSLSSKYMTQGKASAKRTQQES

SEQ ID NO: 13 - protein, 124 aa, Homo sapiens
MSQPDVLLLVQECLKNSDSFIDVDADFHARVPVVVCREKQSGLLCKVSAGNENACLTTKHLTALGKLEPKLVPLVIAFRYWAKLCSIDRPEEGGLPPYVFALMAIFFLQQRKEPLLPVYLGSWV

SEQ ID NO: 14 - protein, 168 aa, Homo sapiens
MEAGSPENKKQRSRPRKPRKTRNEENEQDGDLEGPVIDESVLSTKELLGLQQAEERLKRDCIDRLKRRPRNYPTAKYTCRLCDVLIESIAFAHKHIKEKRHKKNIKEKQEEELLTTLPPPTPSQINAVGIAIDKVVQEFGLHNENLEQRLEIKRIMENVFQHKLPGIF

SEQ ID NO: 15 - protein, 1457 aa, Homo sapiens
MGDTAKPYFVKRTKDRGTMDDDDFRRGHPQQDYLIIDDHAKGHGSKMEKGLQKKKITPGNYGNTPRKGPCAVSSNPYAFKNPIYSQPAWMNDSHKDQSKRWLSDEHTGNSDNWREFKPGPRIPVINRQRKDSFQENEDGYRWQDTRGCRTVRRLFHKDLTSLETTSEMEAGSPENKKQRSRPRKPRKTRNEENEQDGDLEGPVIDESVLSTKELLGLQQAEERLKRDCIDRLKRRPRNYPTAKYTCRLCDVLIESIAFAHKHIKEKRHKKNIKEKQEEELLTTLPPPTPSQINAVGIAIDKVVQEFGLHNENLEQRLEIKRIMENVFQHKLPDCSLRLYGSSCSRLGFKNSDVNIDIQFPAIMSQPDVLLLVQECLKNSDSFIDVDADFHARVPVVVCREKQSGLLCKVSAGNENACLTTKHLTALGKLEPKLVPLVIAFRYWAKLCSIDRPEEGGLPPYVFALMAIFFLQQRKEPLLPVYLGSWIEGFSLSKLGNFNLQDIEKDVVIWEHTDSAAGDTGITKEEAPRETPIKRGQVSLILDVKHQPSVPVGQLWVELLRFYALEFNLADLVISIRVKELVSRELKDWPKKRIAIEDPYSVKRNVARTLNSQPVFEYILHCLRTTYKYFALPHKITKSSLLKPLNAITCISEHSKEVINHHPDVQTKDDKLKNSVLAQGPGATSSAANTCKVQPLTLKETAESFGSPPKEEMGNEHISVHPENSDCIQADVNSDDYKGDKVYHPETGRKNEKEKVGRKGKHLLTVDQKRGEHVVCGSTRNNESESTLDLEGFQNPTAKECEGLATLDNKADLDGESTEGTEELEDSLNHFTHSVQGQTSEMIPSDEEEEDDEEEEEEEEPRLTINQREDEDGMANEDELDNTYTGSGDEDALSEEDDELGEAAKYEDVKECGKHVERALLVELNKISLKEENVCEEKNSPVDQSDFFYEFSKLIFTKGKSPTVVCSLCKREGHLKKDCPEDFKRIQLEPLPPLTPKFLNILDQVCIQCYKDFSPTIIEDQAREHIRQNLESFIRQDFPGTKLSLFGSSKNGFGFKQSDLDVCMTINGLETAEGLDCVRTIEELARVLRKHSGLRNILPITTAKVPIVKFFHLRSGLEVDISLYNTLALHNTRLLSAYSAIDPRVKYLCYTMKVFTKIYKGEKKPEIFVDGWNIYFFDQIDELPTYWSECGKNTESVGQLWLGLLRFYTEEFDFKEHVISIRRKSLLTTFKKQWTSKYIVIEDPFDLNHNLGAGLSRKMTNFIMKAFINGRRVFGIPVKGFPKDYPSKMEYFFDPDVLTEGELAPNDRCCRICGKIGHFMKDCPMRRKVRRRRDQEDALNQRYPENKEKRSKEDKEIHNKYTEREVSTKEDKPIQCTPQKAKPMRAAADLGREKILRPPVEKWKRQDDKDLREKRCFICGREGHIKKECPQFKGSSGSLSSKYMTQGKASAKRTQQES

SEQ ID NO: 16 - protein, 123 aa, Homo sapiens
MSQPDVLLLVQECLKNSDSFIDVDADFHARVPVVVCREKQSGLLCKVSAGNENACLTTKHLTALGKLEPKLVPLVIAFRYWAKLCSIDRPEEGGLPPYVFALMAIFFLQQRKEPLLPVYLGSW

SEQ ID NO: 17 - protein, 615 aa, Homo sapiens
MGDTAKPYFVKRTKDRGTMDDDDFRRGHPQQDYLIIDDHAKGHGSKMEKGLQKKKITPGNYGNTPRKGPCAVSSNPYAFKNPIYSQPAWMNDSHKDQSKRWLSDEHTGNSDNWREFKPGPRIPVINRQRKDSFQENEDGYRWQDTRGCRTVRRLFHKDLTSLETTSEMEAGSPENKKQRSRPRKPRKTRNEENEQDGDLEGPVIDESVLSTKELLGLQQAEERLKRDCIDRLKRRPRNYPTAKYTCRLCDVLIESIAFAHKHIKEKRHKKNIKEKQEEELLTTLPPPTPSQINAVGIAIDKVVQEFGLHNENLEQRLEIKRIMENVFQHKLPDCSLRLYGSSCSRLGFKNSDVNIDIQFPAIMSQPDVLLLVQECLKNSDSFIDVDADFHARVPVVVCREKQSGLLCKVSAGNENACLTTKHLTALGKLEPKLVPLVIAFRYWAKLCSIDRPEEGGLPPYVFALMAIFFLQQRKEPLLPVYLGSWIEGFSLSKLGNFNLQDIEKDVVIWEHTDSAAGDTGITKEEAPRETPIKRGQVSLILDVKHQPSVPVGQLWVELLRFYALEFNLADLVISIRVKELVSRELKDWPKKRIAIEGISKCLSYSPPLFFLKVPV

SEQ ID NO: 18 - protein, 544 aa, Homo sapiens
MGDTAKPYFVKRTKDRGTMDDDDFRRGHPQQDYLIIDDHAKGHGSKMEKGLQKKKITPGNYGNTPRKGPCAVSSNPYAFKNPIYSQPAWMNDSHKDQSKRWLSDEHTGNSDNWREFKPGPRIPVINRQRKDSFQENEDGYRWQDTRGCRTVRRLFHKDLTSLETTSEMEAGSPENKKQRSRPRKPRKTRNEENEQDGDLEGPVIDESVLSTKELLGLQQAEERLKRDCIDRLKRRPRNYPTAKYTCRLCDVLIESIAFAHKHIKEKRHKKNIKEKQEEELLTTLPPPTPSQINAVGIAIDKVVQEFGLHNENLEQRLEIKRIMENVFQHKLPDCSLRLYGSSCSRLGFKNSDVNIDIQFPAIMSQPDVLLLVQECLKNSDSFIDVDADFHARVPVVVCREKQSGLLCKVSAGNENACLTTKHLTALGKLEPKLVPLVIAFRYWAKLCSIDRPEEGGLPPYVFALMAIFFLQQRKEPLLPVYLGSWIEGFSLSKLGNFNLQDIEKDVVIWEHTDSAAGDTGITKEEAPRETPIKRGQVVSSLLCR

SEQ ID NO: 19 - DNA, 2365 bp, Caenorhabditis elegans; identical to SEQ ID NO: 1

SEQ ID NO: 20 - protein, 227 aa, Caenorhabditis elegans
MSTVVSEGRNDGNNRYSPQDEVEDRLPDVVDNRLTENMRVPSFERLPSPTPRYFGSCKWFNVSKGYGFVIDDITGEDLFVHQSNLNMQGFRSLDEGERVSYYIQERSNGKGREAYAVSGEVEGQGLKGSRIHPLGRKKAVSLRCFRCGKFATHKAKSCPNVKTDAKVCYTCGSEEHVSSICPERRRKHRPEQVAAEEAEAARMAAEKSSPTTSDDDIREKNSNSSDE

SEQ ID NO: 21 - DNA, 4014 bp, Homo sapiens
gtgcgggggaagatgtagcagcttcttctccgaaccaaccctttgccttcggacttctccggggccagcagccgcccgaccaggggcccggggccacgggctcagccgacgaccatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattgagccacaatgggtgggggctattcttttgctatcaggaagttttgaggagcaggcagagtggagaaagtgggaatagggtgcattggggctagttggcactgccatgtatctcaggcttgggttcacaccatcaccctttcttccctctaggtggggggaaagggtgagtcaaaggaactccaaccatgctctgtccaaatgcaagtgagggttctgggggcaaccaggaggggggaatcaccctacaacctgcatactttgagtctccatccccagaatttccagcttttgaaagtggcctggatagggaagttgttttccttttaaagaaggatatataataattcccatgccagagtgaaatgattaagtataagaccagattcatggagccaagccactacattctgtggaaggagatctctcaggagtaagcattgtttttttttcacatcttgtatcctcatacccacttttgggatagggtgctggcagctgtcccaagcaatgggtaatgatgatggcaaaaagggtgtttgggggaacagctgcagacctgctgctctatgctcacccccgccccattctgggccaatgtgattttatttatttgctcccttggatactgcaccttgggtcccactttctccaggatgccaactgcactagctgtgtgcgaatgacgtatcttgtgcattttaactttttttccttaatataaatattctggttttgtatttttgtatattttaatctaaggccctcatttcctgcactgtgttctcaggtacatgagcaatctcagggatagccagcagcagctccaggtctgcgcagcaggaattactttttgttgtttttgccaccgtggagagcaactatttggagtgcacagcctattgaactacctcatttttgccaataagagctggcttttctgccatagtgtcctcttgaaaccccctctgccttgaaaatgttttatgggagactaggttttaactgggtggccccatgacttgattgccttctactggaagattgggaattagtctaaacaggaaatggtggtacacagaggctaggagaggctgggcccggtgaaaaggccagagagcaagccaagattaggtgagggttgtctaatcctatggcacaggacgtgctttacatctccagatctgttcttcaccagattaggttaggcctaccatgtgccacagggtgtgtgtgtgtttgtaaaactagagttgctaaggataagtttaaagaccaatacccctgtacttaatcctgtgctgtcgagggatggatatatgaagtaaggtgagatccttaacctttcaaaattttcgggttccagggagacacacaagcgagggttttgtggtgcctggagcctgtgtcctgccctgctacagtagtgattaatagtgtcatggtagctaaaggagaaaaagggggtttcgtttacacgctgtgagatcaccgcaaacctaccttactgtgttgaaacgggacaaatgcaatagaacgcattgggtggtgtgtgtctgatcctgggttcttgtctcccctaaatgctgccccccaagttactgtatttgtctgggctttgtaggacttcactacgttgattgctaggtggcctagtttgtgtaaatataatgtattggtctttctccgtgttctttgggggttttgtttacaaacttctttttgtattgagagaaaaatagccaaagcatctttgacagaaggttctgcaccaggcaaaaagatctgaaacattagtttggggggccctcttcttaaagtggggatcttgaaccatcctttcttttgtattccccttcccctattacctattagaccagatcttctgtcctaaaaacttgtcttctaccctgccctcttttctgttcacccccaaaagaaaacttacacacccacacacatacacatttcatgcttggagtgtctccacaactcttaaatgatgtatgcaaaaatactgaagctaggaaaaccctccatcccttgttcccaacctcctaagtcaagaccattaccatttctttctttcttttttttttttttttaaaatggagtctcactgtgtcacccaggctggagtgcagtggcatgatcggctcactgcagcctctgcctcttgggttcaagtgattctcctgcctcagcctcctgagtagctgggatttcaggcacccgccacactcagctaatttttgtatttttagtagagacggggtttcaccatgttgtccaggctggtctggaactcctgacctcaggtgatctgcccaccttggcttcccaaagtgctgggattacaggcatgagccaccatgctgggccaaccatttcttggtgtattcatgccaaacacttaagacactgctgtagcccaggcgcggtggctcacacctgtaatcccagcactttggaaggctgaggcgggcggatcacaaggtcacgagttcaaaactatcctggccaacacagtgaaaccccgtctctactaaaatacaaaaaaattagccgggtgtggtggtgcatgcctttagtcctagctattcaggaggctgaggcaggggaatcgcttgaacccgagaggcagaggttgcagtgagctgagatcgcaccactgcactccagcctggttacagagcaagactctgtctcaaacaaaacaaaacaaaacaaaaacacactactgtattttggatggatcaaacctccttaattttaatttctaatcctaaagtaaagagatgcaattgggggccttccatgtagaaagtggggtcaggaggccaagaaagggaatatgaatgtatatccaagtcactcaggaacttttatgcaggtgctagaaactttatgtcaaagtggccacaagattgtttaataggagacgaacgaatgtaactccatgtttactgctaaaaaccaaagctttgtgtaaaatcttgaatttatggggcgggagggtaggaaagcctgtacctgtctgtttttttcctgatccttttccctcattcctgaactgcaggagactgagcccctttgggctttggtgaccccatcactggggtgtgtttatttgatggttgattttgctgtactgggtacttcctttcccattttctaatcattttttaacacaagctgactcttcccttcccttctcctttccctgggaaaatacaatgaataaataaagacttattggtacgcaaactgtca

SEQ ID NO: 22 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 23 - RNA, 21 nt, Caenorhabditis elegans
cuaugcaauuuucaccuuacc

SEQ ID NO: 24 - RNA, 22 nt, Caenorhabditis elegans
cuaugcaauuuucaccuuaccu

SEQ ID NO: 25 - RNA, 23 nt, Caenorhabditis elegans
cuaugcaauuuucaccuuaccuu

SEQ ID NO: 26 - DNA, 32 bp, Caenorhabditis elegans
accggtgaactatgcaattttcaccttaccgg